(19) United States
(12) Patent Application Publication
(10) Pub. No.: US 2007/0231393 A1
(43) Pub. Date: Oct. 4, 2007

(54) System and Device for Magnetic Drug Targeting with Magnetic Drug Carrier Particles

(75) Inventors: James A. Ritter, Lexington, SC (US); Armin D. Ebner, Lexington, SC (US); Charles E. Holland, Cayce, SC (US)

Correspondence Address: NEEDLE & ROSENBERG, P.C., SUITE 1000, 999 PEACHTREE STREET, ATLANTA, GA, 30309-3915, US

(73) Assignee: UNIVERSITY OF SOUTH CAROLINA, Columbia, SC (US)

(21) Appl. No.: 11/596,820

(22) PCT Filed: May 19, 2005
(86) PCT No.: PCT/US05/17644

§ 371 Date: May 8, 2007

Related U.S. Application Data
(60) Provisional application No. 60/572,370, filed on May 19, 2004.
(60) Provisional application No. 60/572,439, filed on May 19, 2004.

(51) Int. Cl. A61K 47/02; A61M 37/00; A61N 2/00

(52) U.S. Cl. 424/489; 436/526; 600/12; 604/890.1; 623/1.11

(57) ABSTRACT

Disclosed are methods of positioning a magnetic drug carrier particle within the body of a subject comprising placing an article within the body of the subject or external to the body of a subject; inserting a magnetic drug carrier particle into the body of the subject, and applying an external magnetic field to the article, thereby causing the magnetic drug carrier particle to be attracted to the article. Also disclosed are articles, systems, and kits that can be used in the disclosed methods.

### CROSS REFERENCE TO RELATED APPLICATIONS

This application claims the benefit of priority to U.S. Provisional Application No. 60/572,370, filed May 19, 2004, and U.S. Provisional Application No. 60/572,439, filed May 19, 2004. U.S. Provisional Application Nos. 60/572,370 and 60/572,439 are incorporated by reference herein in their entireties.

### ACKNOWLEDGMENT OF GOVERNMENT FUNDING

The research described herein was supported by the National Science Foundation under grant No. CTS-0314157. The U.S. government has certain rights in this invention.

### FIELD

The disclosed subject matter, in one aspect, generally relates to a therapeutic treatment system, and, more particularly, to therapeutic targeted drug delivery with magnetic devices and magnetic fields.

### BACKGROUND

Typically, when a drug is taken into the body (orally, intravenously, etc.) to treat a medical condition, only a very small percentage of it actually reaches and treats the intended target site. In some cases this could be as little as 2%. This can be a wasteful use of a drug, especially when some medicines, e.g., the flu vaccine, are desperately needed by a large fraction of the population and 40 to 50 times the required dose must be administered to be effective. As such, much research has been devoted to minimizing drug scarcity by ensuring that more of a dose actually reaches the target site and carries out the medical treatment for which it was designed.

One approach to increase targeting efficiency has been to use magnetic drug carrier particles (MDCPs)(see e.g., Hafeli, Int. J. Pharmaceutics 277:19-24, 2004); Shinkai, J. Bioscience Bioengineering 94:606-613, 2002). The ability of these particles to be attracted to a magnetic source makes them candidates for localized magnetic drug targeting (MDT) systems. Many studies have shown that it is indeed possible for these magnetic particles, even when carrying drugs or radioactive species, to be magnetically retained and localized at specified locations in the body (Lübbe, et al., J. Surg Res 95:200-203, 2001; Goodwin, et al., J. Magnetism Magnetic Materials 194:132-139, 1999).

Most of the articles that discuss various MDT technologies and approaches employ an external magnet positioned near a target site that is located at some depth below the skin to attract and retain the MDCPs at the site. The purpose of the magnet is to impart an attractive force on the MDCP that is large enough to overcome any hydrodynamic force associated with blood flow in the circulatory system. Even though the hydrodynamic force is the only major force the MDCPs are exposed to, its magnitude varies widely, due to the large disparity in blood velocities ranging from less than 0.1 cm/s in capillaries to over 1 m/s in large arteries (Popel, Network models of peripheral circulation, in: Handbook of Bioengineering, C. Skalak and S. Chien (Eds.), McGraw-Hill, N.Y., 1987, Ch 20; Berger, et al., (Eds.), Introduction to Bioengineering, Oxford University Press, New York, 1996; Saltzman, Drug Delivery-Engineering Principles for Drug Delivery, Oxford University Press, New York, 2001; Ghassabian, et al., Int. J. Pharm., 130(1):49-55, 1996; Goldsmith and Turitto, Thrombosis Haemistasis 55:415, 1986).

Certain limitations have become apparent with previous MDT approaches. First, the retention of the MDCPs even in dense, muscular tissue is quite low due to the inherently weak nature of the magnetic force. The hydrodynamic force associated with capillary blood flow in this kind of tissue, with velocities less than 0.1 cm/s, still dominates the magnetic force, even in the most favorable situation, i.e., when the permanent magnet is located very close to the disease site, which is rarely the case. Hence, the depth of the target site is another limitation associated with the traditional MDT approach. Sites that are more than a few centimeters deep in the body are difficult to target, partially because the strength of the magnetic field generated from a permanent magnet decreases sharply with distance (Goodwin, et al., J. Magnetism Magnetic Materials, 194:132-139, 1999).

Targeted drug delivery is an important goal of modern medical pharmaco- and radiotherapy. And there is currently a need for methods and compositions that seek to avoid systemic drug side effects by using smaller amounts of medication, focusing delivery to a desired region, and controlling the onset and termination of drug action at a target site. The methods and compositions disclosed herein meet these and other needs.

### SUMMARY

In accordance with the purposes of the disclosed materials, compounds, compositions, and methods, as embodied and broadly described herein, the disclosed subject matter, in one aspect, relates to compounds and compositions and methods for preparing and using such compounds and compositions. In another aspect, disclosed herein is the use of a device reactive to an external field generator to allow for targeted application of at least one magnetic carrier particle, such as, for example, a magnetic drug carrier particle, to a targeted location within or without a body of an organism. In another aspect, disclosed herein is the use of the disclosed materials, compounds, compositions and methods for therapeutic targeted drug delivery. In a further aspect are devices, systems comprising such devices, methods of using such devices, and kits

The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DETAILED DESCRIPTION OF THE FIGURES

The accompanying Figures, which are incorporated in and constitute a part of this specification, illustrate several aspects and, together with the description, illustrate the disclosed compositions and methods.

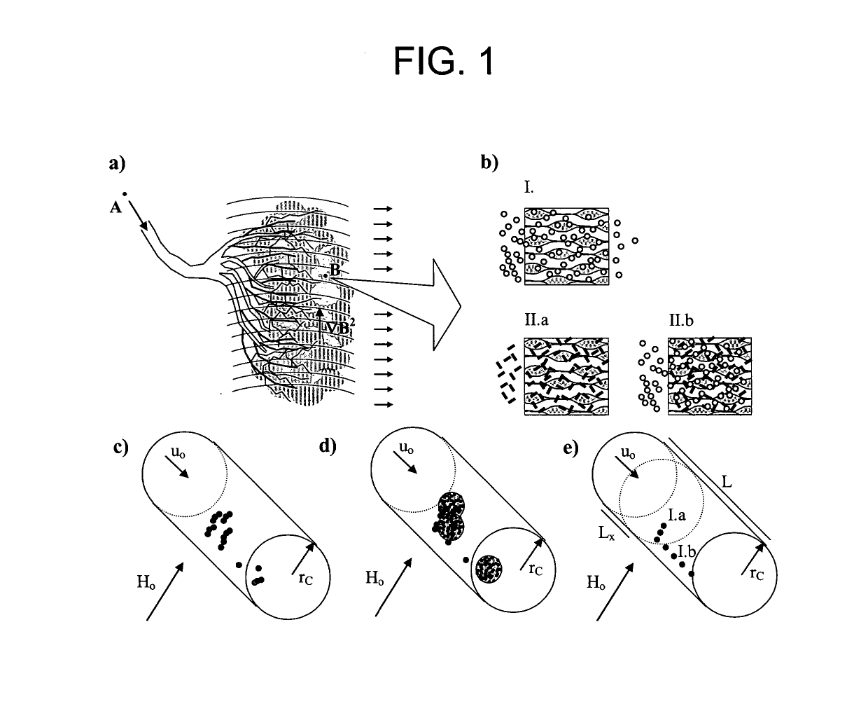

FIGS. 1(a-e) are schematics illustrating the concept of the use of the magnetic seeds for magnetic drug targeting (“MDT”). FIG. 1(a) is a macroscopic view of a zone where drug targeting is required. The zone is under the influence of a magnetic field produced by an external magnet. It is being fed from left to right by the artery on the left at point A, which branches into arterioles and capillaries (gray zone). FIG. 1(b) is a microscopic view of a capillary system at point B in FIG. 1(a) showing the two alternative procedures for collecting magnetic drug carrier particles (“MDCPs”): I) where the MDCPs are partially retained solely based on the strength and gradients of the magnetic field from the external magnet, and II) where the disclosed magnetizable seeds are more easily retained at the affected zone by the external magnet field (II.a) and are then used to more effectively collect the MDCPs moving downstream (II.b). FIG. 1(c) shows seeds of radius r_{nd} dispersed along the capillary and forming magnetically aligned filaments. FIG. 1(d) is a schematic showing how MDCPs are retained by the seeds. FIG. 1(e) is a schematic of the theoretical control volume used for mathematical evaluation that represents a capillary containing an aligned filament comprised of seeds (I.a) or individual seeds (I.b). The blood flow enters with a parabolic profile with average velocity uₒ from the upstream end of the capillary, and the external magnetic field lies in a plane perpendicular to the axis of the capillary inclined at angle α with respect to a horizontal line contained in the same plane.

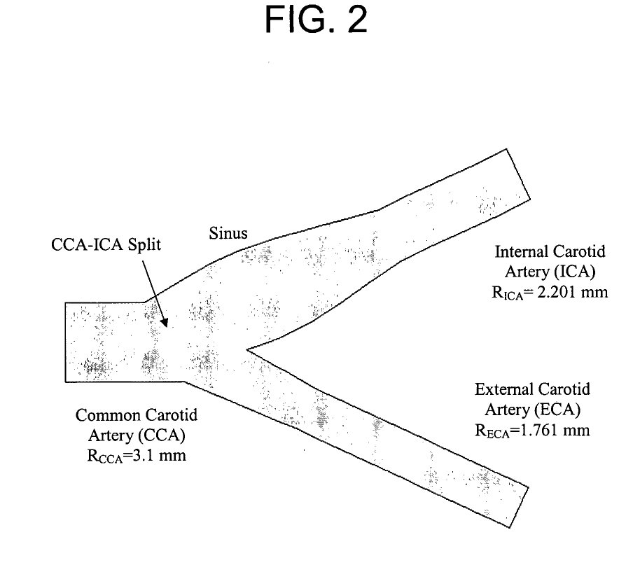

FIG. 2 is a schematic of a carotid artery bifurcation showing the common carotid artery (CCA) and the split into the internal carotid artery (ICA) and the external carotid artery (ECA). The schematic is a modification of the carotid artery bifurcation reported by Bharvadaj (Bharvadaj, et al., J. Biomechanics 15(5):363-378, 1982) and later found at Ma (Ma, et al., J. Biomechanics 30(6):565-571, 1997).

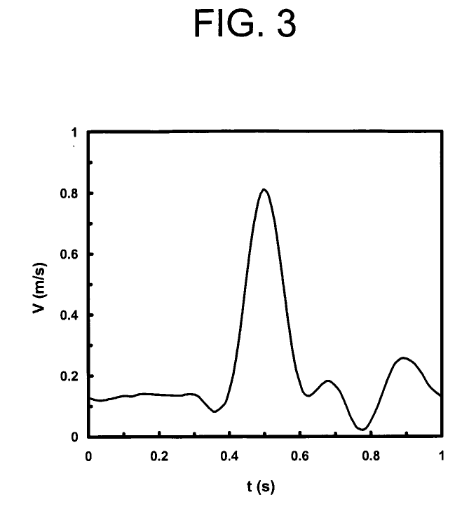

FIG. 3 is graph showing an average inlet velocity (m/s) at the entrance of the common carotid artery as a function of time (s) during one pulse.

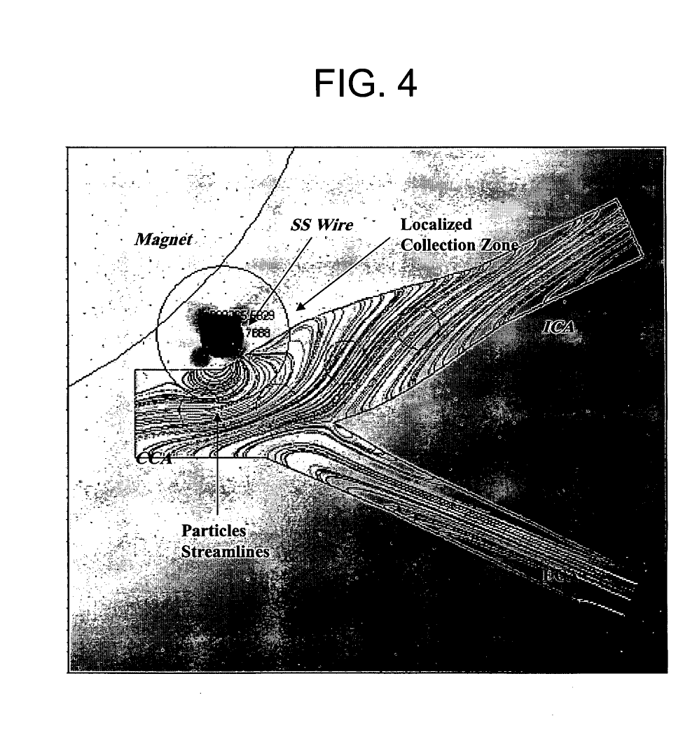

FIG. 4 is a schematic of the carotid artery studied in a FEMLAB computer model of therapeutic treatment system disclosed herein. The schematic shows a magnet with a radius of 20 times that of the common carotid artery (CCA) and a wire with a radius half of that of the CCA. The target zone is defined as 3 times the wire radius. The streamlines show the particle trajectories and are used to determine the percentage of particles collected.

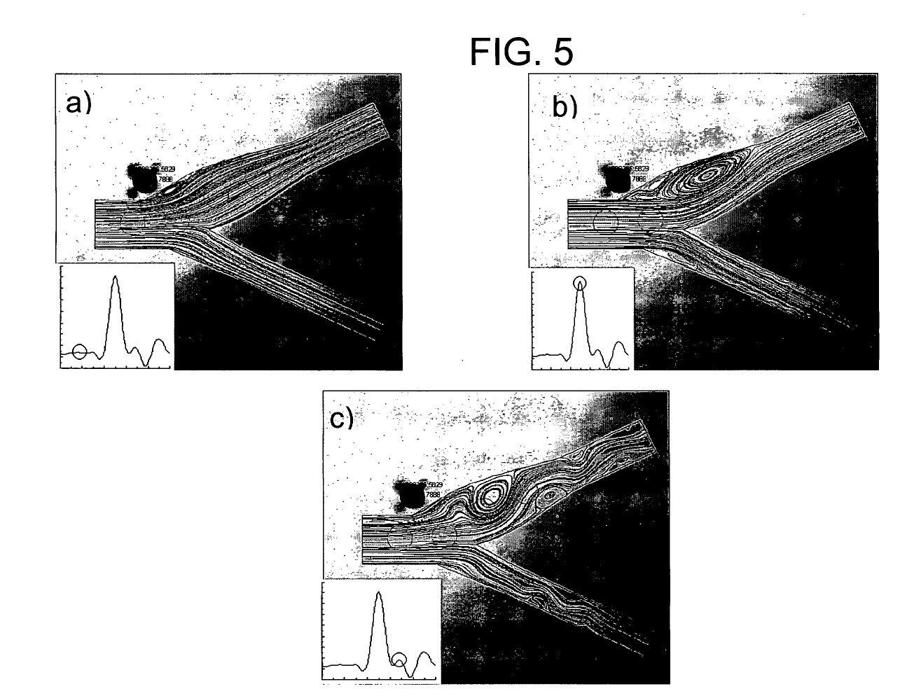

FIGS. 5(a-c) are schematics of the carotid artery from a FEMLAB computer model of fluid streamlines when no magnetic force is applied at different points during the pulsatile flow. FIG. 5(a) shows the particle trajectories when the velocity is at diastolic point (velocity is about 0.2 m/s), (b) at the systolic point (maximum velocity is about 0.9 m/s), and (c) at the end of the systolic point (velocity is about 0.3 m/s) (see inset graphs referring to FIG. 3).

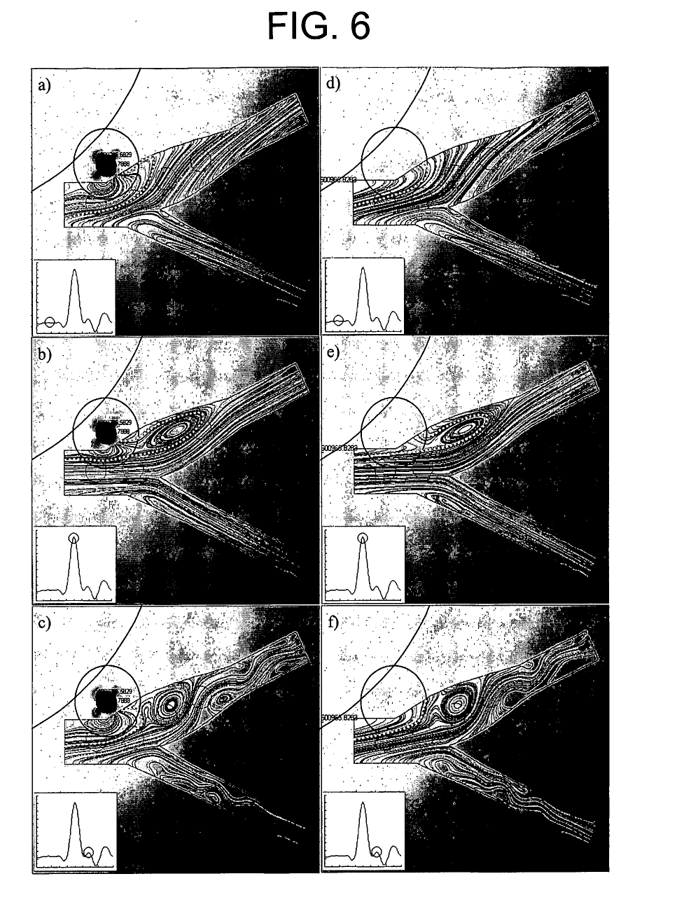

FIGS. 6(a-f) are schematics of the carotid artery from a FEMLAB computer model showing the retention of particles at the CCA-ICA split of the carotid artery at different times. The results shown are for magnetic drug carrier particles (MDCPs) (χₚ=1000, M_{p,s}=480,000) with radius (Rₚ) of 50 μm and magnetite content (x_{fm}) of 0.2. FIGS. 6(a) to 6(c) show the area of collection (white dashed line) for the case of a permanent magnet (Mₙ=1,200,000 A/m, Rₘ=6.2 cm) combined with a wire (χ_{w}=1000, M_{w,s}=1,650,000 A/m, R_{w}=1.55 mm). FIGS. 6(d) to 6(f) show the area of collection for the case of a permanent magnet only (Mₘ=1,200,000 A/m, Rₘ=6.2 cm). For time reference see inset referring to FIG. 3.

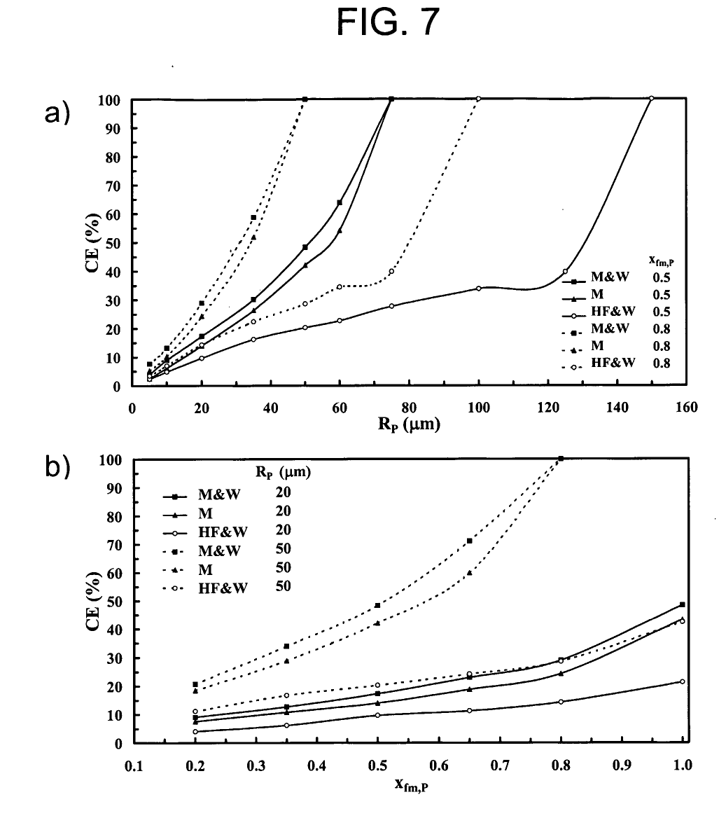

FIGS. 7(a-b) are graphs showing the effect of the particle radius (Rₚ) and magnetite content (x_{fm}) on the collection efficiency (CE) for a magnet and wire, permanent magnet alone, and homogenous field (H₀=537,780 A/m) combined with a wire. FIG. 7(a) shows the effect of the particle radius (Rₚ) at different magnetite content (x_{fm}=0.5, x_{fm}=0.8). The particle size is changed until 100% collection is reached. FIG. 7(b) shows the effect of MDCP magnetite content (x_{fm}) on the collection efficiency (CE) at different particle sizes (Rₚ=20 μm, Rₚ=50 μm).

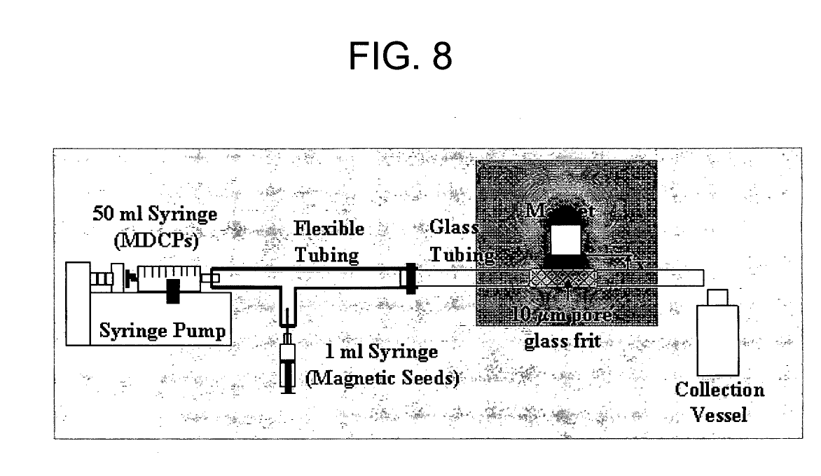

FIG. 8 is a schematic of the experimental setup for in vitro testing of the ferromagnetic seeds concept for MDT.

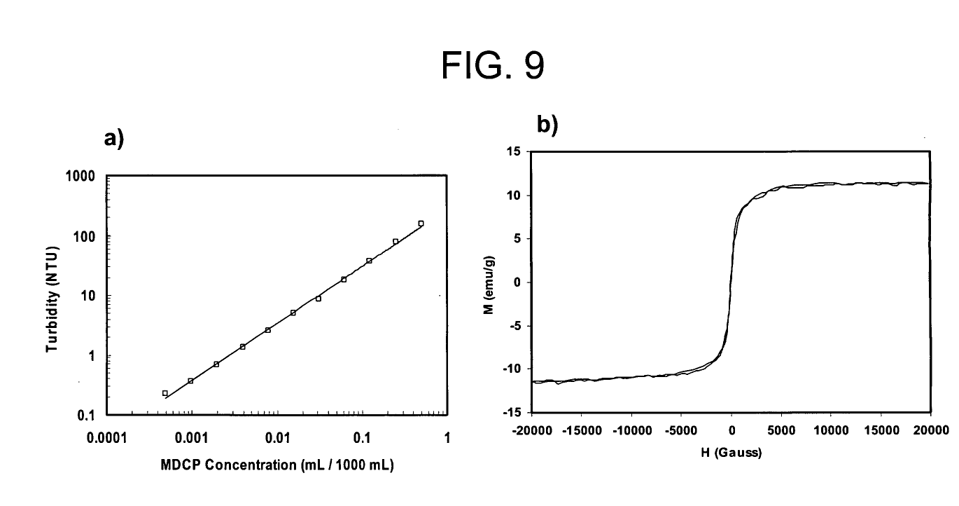

FIGS. 9(a-b) are graphs showing the typical concentration versus turbidity calibration plot (FIG. 9(a)) and a magnetization plot for superparamagnetic microspheres (FIG. 9(b)) (Rp=1.165 μm, 20 wt % magnetite, Bangs Laboratories, Inc.). The calibration plot was measured by diluting a given volume of the as purchased sample containing 10 wt % of suspended microspheres. The magnetization was obtained from a dry sample of these microspheres.

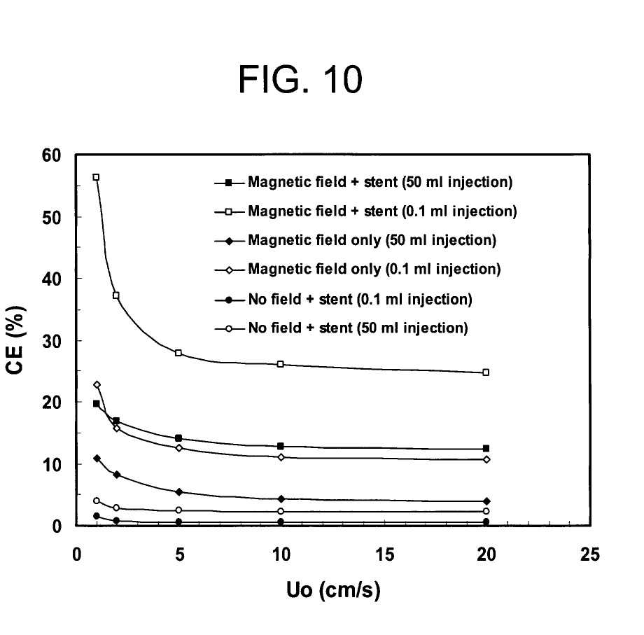

FIG. 10 is a graph showing typical experimental results depicting the collection efficiency (CE) as a function of velocity using a setup that is similar to that shown in FIG. 8, where a 1 cm section consisting of 1 mm ID glass tubing containing a spring shaped (stent) ferromagnetic wire. The wire was replaced with the fritted glass section. The wire (SS 430, Mₛₐₜ=1340 kA/m) thickness was 125 μm and its luminal diameter was of 0.75 mm. The magnetic field source was the 0.6 T, 50×50×25 mm cube magnet, which was attached to the glass tubing (i.e., x=0). In all cases the same total amount (equivalent to 2.5 mg of dry sample) of magnetic particles (Rₚ=1.165 μm, 20 wt % magnetite, Bangs Laboratories Inc., Fisher, Ind.) was administered, either continuously through a full 50 ml syringe or instantly in 0.1 ml doses that were added sidewise using a 1 ml syringe.

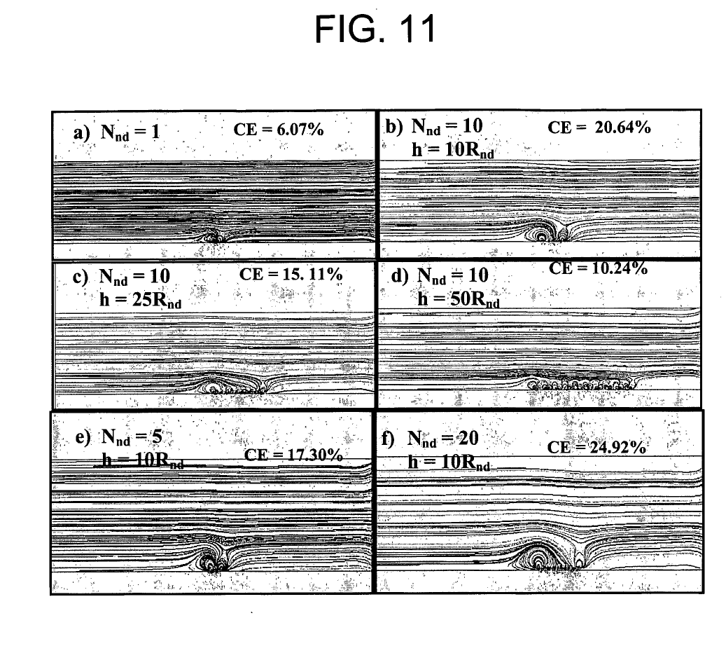

FIGS. 11(a-f) are schematics from a FEMLAB computer model showing collection efficiencies of MDCPs (Rₚ=1 μm, 40 wt % magnetite (Mₛₐₜ=480 kA/m) by (a) a single spherical magnetic seed, and by (b-f) different arrays (by varying the number of seeds N_{nd} and the interseed separation, h, of spherical magnetic seeds (R_{nd}=20 nm, Mₛₐₜ=1350 kA/m) under a homogeneous external field of 1.5 T and a mean blood velocity of 0.1 cm/s using the 2-D streamline analysis approach outlined in the text and elsewhere (see Ritter, et al., J. Magn. Magn. Mater., 280:184-201, 2004; Chen, et al., J. Magn. Magn. Mater., 284:181-194, 2004; Aviles, et al., J. Magn. Magn. Mater., 293:605-615 (20054); Chen, et al., J. Magn. Magn. Mater., 293:616-632, 2005).

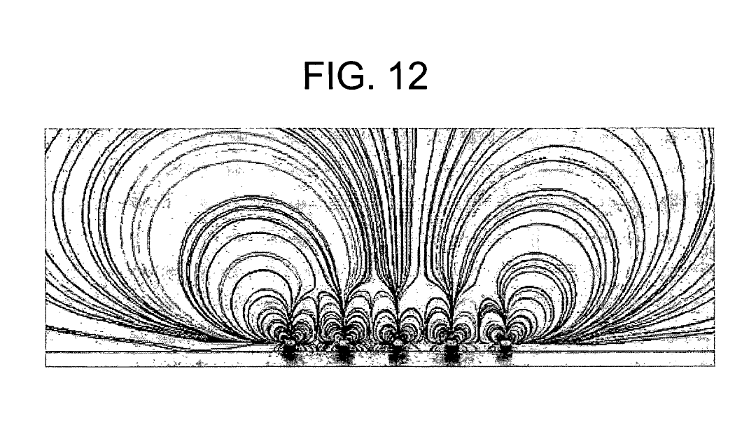

FIG. 12 is a magnified view of FIG. 11(e), which depicts the streamlines corresponding to collected MDCPs.

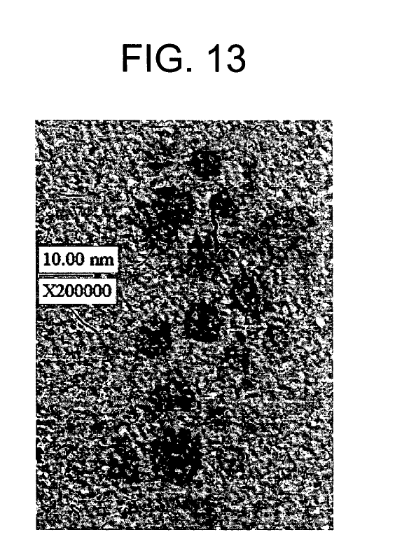

FIG. 13 is a micrograph of an iron oxide colloid obtained by direct sonochemical decomposition of Fe(CO)₅ in the presence of oleic acid (see Shafi, et al., Thin Solid Films, 318:38, 1998; Prozorov, et al., Nanostr. Mater., 12:669, 1999).

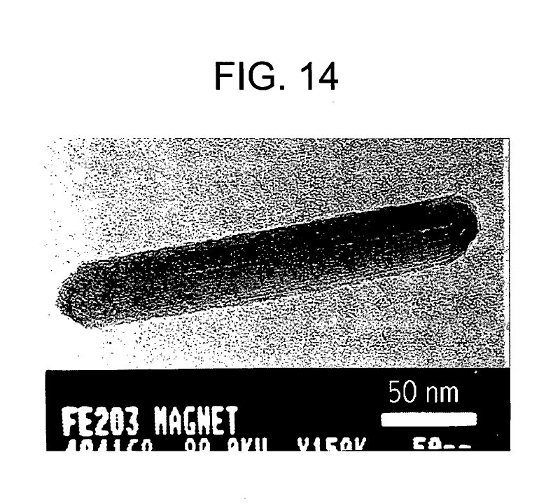

FIG. 14 is a micrograph of Fe₂O₃ ferromagnetic nanoparticle obtained by using sonochemical synthesis in a magnetic field (see Prozorov, et al., J. Phys. Chem. B 102, 10165, 1998).

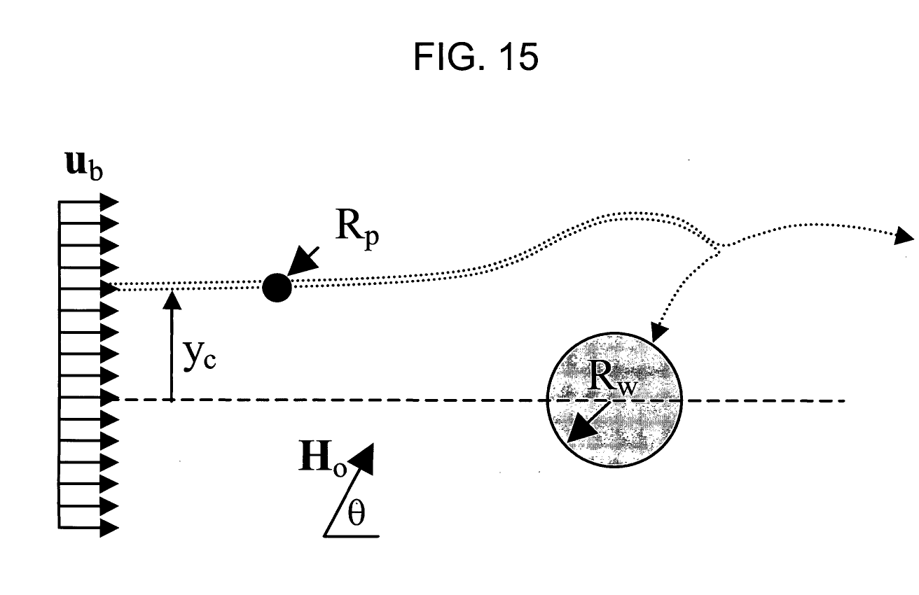

FIG. 15 is a schematic of a ferromagnetic wire of radius R_{w} that is placed perpendicular to the plane of the figure, facing blood that is moving from left to right with velocity U_{b}, and under an applied magnetic field μₒHₒ that is resting in the plane of the figure and pointing in a direction defined by angle θ. The blood transports the ferromagnetic MDCPs of radius Rₚ past the wire that has a capture cross-section y_{c}.

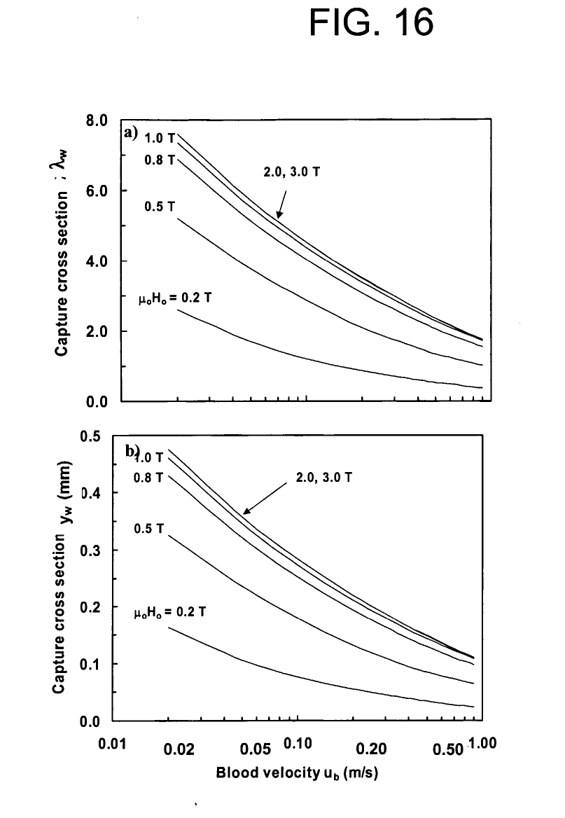

FIGS. 16(a-b) are graphs showing the effect of (b) blood velocity (u_{b}) and (a) magnetic field strength (μₒHₒ) on the dimensionless and dimensional capture cross-sections of spherical MDCPs made of 100% iron (xₚ=100 wt %) and with Rₚ=1 μm that are collected by a magnetically energized wire made of iron (R_{w}=62.5 μm) and placed perpendicular to the liquid flow. The remaining parameters are given in Tables 2, 3, and 4 below.

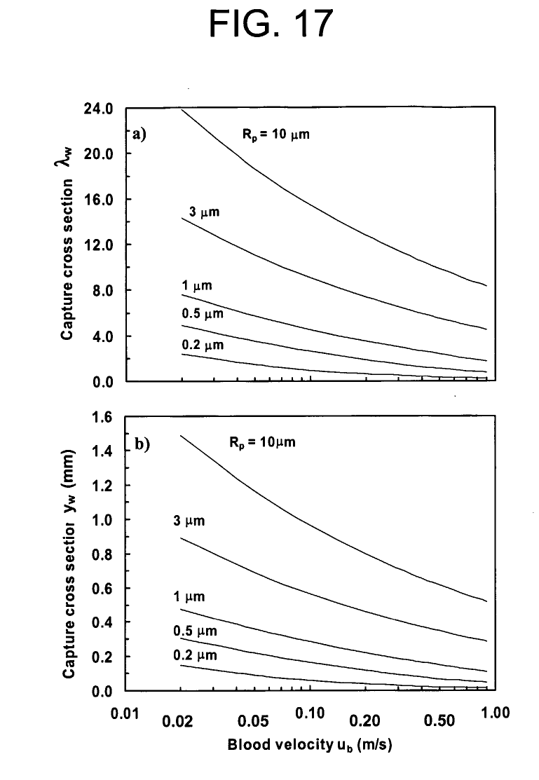

FIGS. 17(a-b) are graphs showing the effect of (b) blood velocity (u_{b}) and (a) MDCP size (Rₚ) on the dimensionless and dimensional capture cross-sections of spherical MDCPs made of 100% iron (xₚ=100 wt %) that are collected by a magnetically energized wire made of iron (R_{w}=62.5 μm) and placed perpendicular to the liquid flow and for a magnetic field strength (μₒHₒ) of 2.0 T. The results corresponding to a MDCP with Rₚ=10 μm and porosity (εₚ) of 0.4 assumes that this particle consists of an agglomeration of MDCPs. The remaining parameters are given in Tables 2, 3, and 4 below.

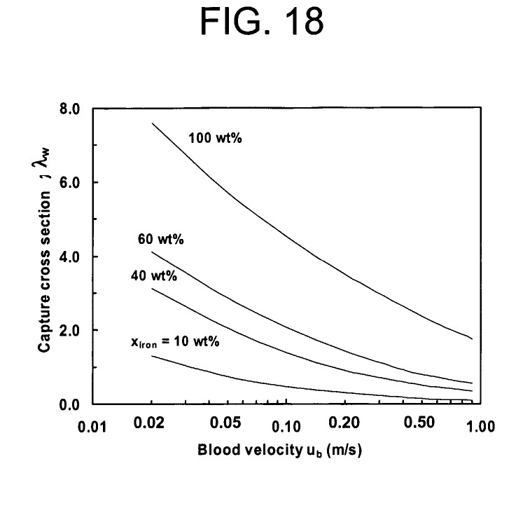

FIG. 18 is a graph showing the effect of blood velocity (u_{b}) and MDCP iron content (xₚ) on the dimensionless and dimensional capture cross-sections of spherical MDCPs with Rₚ=1 μm that are collected by a magnetically energized wire made of iron (R_{w}=62.5 μm) and placed perpendicular to the liquid flow and for a magnetic field strength (μₒHₒ) of 2.0 T. The remaining parameters are given in Tables 2, 3, and 4 below.

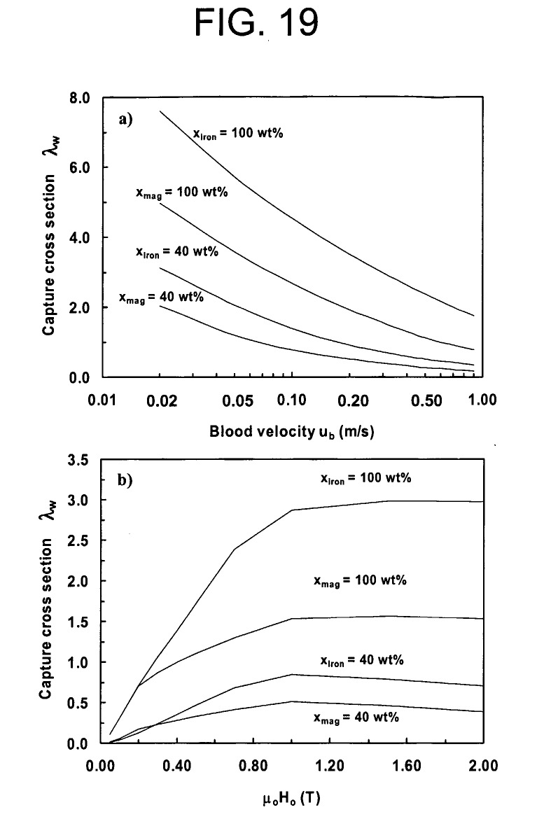

FIGS. 19(a-b) are graphs showing the effect of (a) blood velocity (u_{b}) for a magnetic field strength (μₒHₒ) of 2.0 T and (b) magnetic field strength (μₒHₒ) for a blood velocity of 0.3 m/s on the dimensionless capture cross-section of spherical MDCPs (Rₚ=1 μm) containing different amounts (xₚ) of iron or magnetite that are collected by a magnetically energized wire made of iron (R_{w}=62.5 μm) and placed perpendicular to the liquid flow. The remaining parameters are given in Tables 2, 3, and 4 below.

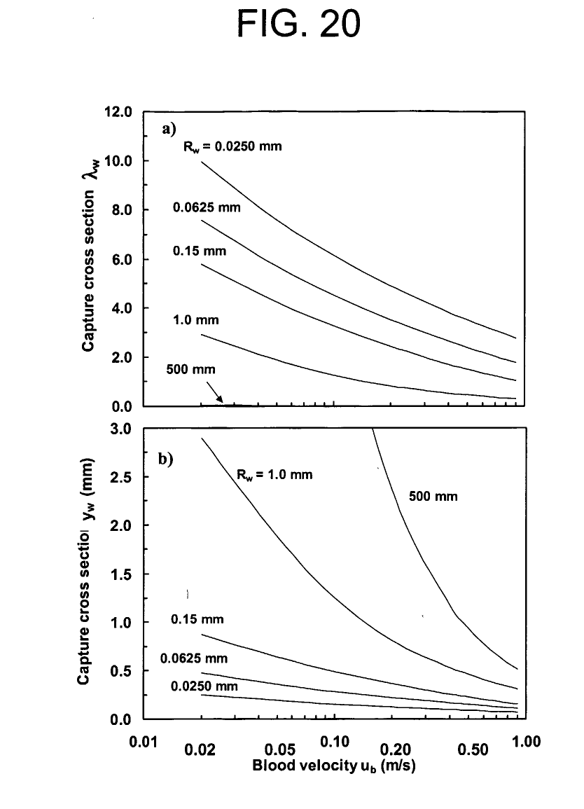

FIGS. 20(a-b) are graphs showing the effect of (b) blood velocity (u_{b}) and (a) wire size (R_{w}) on the dimensionless and dimensional capture cross-sections of spherical MDCPs made of 100% iron (xₚ=100 wt %) and with Rₚ=1 μm that are collected by a magnetically energized wire made of iron placed perpendicular to the liquid flow and for a magnetic field strength (μₒHₒ) of 2.0 T. The remaining parameters are given in Tables 2, 3, and 4 below.

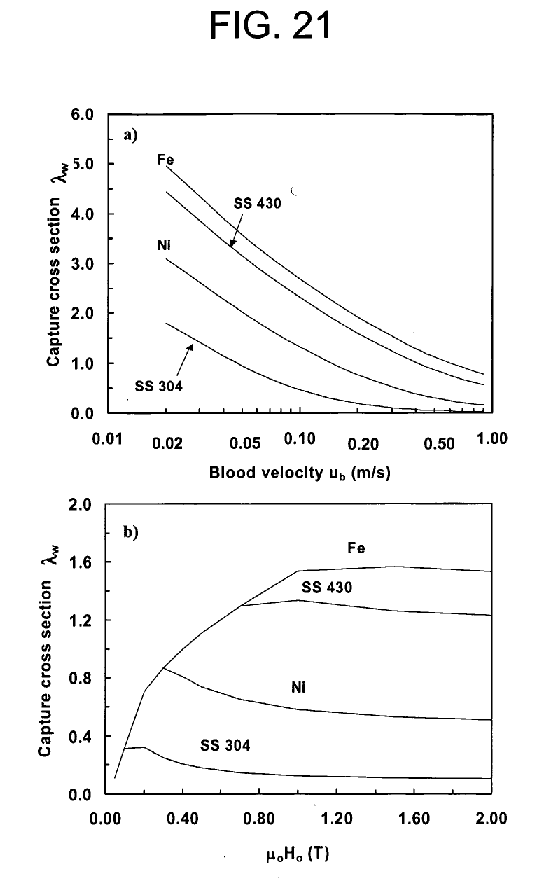

FIGS. 21(a-b) are graphs showing the effect of (a) blood velocity (u_{b}) for a magnetic field strength (μₒHₒ) of 2.0 T and (b) magnetic field strength (μₒHₒ) for a blood velocity of 0.3 m/s on the dimensionless capture cross-section of spherical MDCPs made of 100% magnetite (xₚ=100%) and with Rₚ=1 μm that are collected by a magnetically energized wire (R_{w}=62.5 μm) made of either Fe, Ni, 430 SS or 304 SS, and placed perpendicular to the liquid flow. The remaining parameters are given in Tables 2, 3, and 4 below.

### DETAILED DESCRIPTION

The materials, compounds, compositions, articles, devices, and methods described herein can be understood more readily by reference to the following detailed description of specific aspects of the disclosed subject matter and the Examples included herein and to the Figures.

Before the present materials, compounds, compositions, articles, devices, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims which follow, reference will be made to a number of terms which shall be defined to have the following meanings:

Throughout the specification and claims the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a seed” includes mixtures of two or more such seeds, reference to “an article” includes mixtures of two or more such articles, reference to “the particle” includes mixtures of two or more such particles, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed then “less than or equal to 10” as well as “greater than or equal to 10” is also disclosed. It is also understood that throughout the application, data are provided in a number of different formats, and that this data, represent endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

“Optional” or “optionally” means that the subsequently described event or circumstance can or cannot occur, and that the description includes instances where the event or circumstance occurs and instances where it does not.

“Treatment” or “treating” means to administer a composition to, article a device in, or perform a procedure on a subject or a system with an undesired condition (e.g., restenosis or cancer). The condition can include a disease. “Prevention” or “preventing” means to administer a composition to, article a device in, or perform a procedure on a subject or a system at risk for the condition. The condition can include a predisposition to a disease. The effect of the administration, implantation, or performing a procedure (for treating and/or preventing) can be, but need not be limited to, the cessation of a particular symptom of a condition, a reduction or prevention of the symptoms of a condition, a reduction in the severity of the condition, the complete ablation of the condition, a stabilization or delay of the development or progression of a particular event or characteristic, or minimization of the chances that a particular event or characteristic will occur. It is understood that where treat or prevent are used, unless specifically indicated otherwise, the use of the other word is also expressly disclosed.

By “subject” is meant an individual. The subject can be a mammal such as a primate or a human. The term “subject” can also include domesticated animals including, but not limited to, cats, dogs, etc., livestock (e.g., cattle, horses, pigs, sheep, goats, etc.), and laboratory animals (e.g., mouse, rabbit, rat, guinea pig, etc.).

Disclosed herein, in one aspect, is the use of high gradient magnetic separation (HGMS) in MDT systems (see Ritter, et al., J. of Magn. Magn. Mat., 280:184-201, 2004; Forbes, et al., IEEE Trans Magnets 39:3372-3377, 2003). HGMS is based on the principle that ferromagnetic materials, and many other kinds of magnetic materials including, but not limited to, paramagnetic, superparamagnetic, anti-ferromagnetic, and ferrimagnetic materials, when placed in a magnetic field produce an additional external magnetic field close to its surroundings. Thus, higher magnetic fields can be created inside the body with the introduction of articles comprising ferromagnetic materials (e.g., wires, catheters, stents, seeds, and the like, and as are described herein) near the site being targeted with magnetic drug carrier particles (MDCPs).

In one methodology of the disclosed methods and articles in an extravascular application, a transdermal, ferromagnetic wire is placed or positioned near a diseased and treated carotid bifurcation. The carotid arteries are the main arteries that provide blood to the brain. These arteries are affected by atherosclerosis causing stenosis or narrowing of the artery, a condition commonly referred to as carotid artery disease. It is believed that 20-30% of strokes are due to carotid artery disease (Simon and Zago, Cardiology Rounds 5,5, 2001). Treatment of carotid artery disease consists of the revascularization of the artery through carotid endarterectomy, balloon angioplasty and stenting. Restenosis is the re-narrowing of the artery, after revascularization, which is quite common and usually requires further invasive or some kind of drug therapy for treatment (Cremonsi, et al., Ital Heart J. 1:801-809, 2000; Gershlick, Atherosclerosis 160:259-271, 2002; Szabo, et al., Eur. J. Endovasc. Surg 27:537-539, 2004).

The MDT system disclosed herein can provide a mildly invasive technique when compared with conventional angioplasty or endarterectomy procedures. A wire can be implanted under the skin, next to the carotid artery and used to collect and retain MDCPs at this site to treat restenosis using an external magnet (Gershlick, Atherosclerosis 160:259-271, 2002). Therefore, the system disclosed herien allows for the use of a ferromagnetic wire implanted under the skin next to, or adjacent, the carotid artery to assist in the collection of MDCPs at this targeted location using an external magnet. Several MDT systems are disclosed herein, for example those that use a permanent magnet combined with an article, such as a wire or seed, and those that use a magnetic field combined with an article. The effect of the MDCP size and its magnetic material content are disclosed herein.

In alternative aspects, disclosed herein are methods and compositions that can minimize the dose and thus side effects and toxicity of a drug by maximizing both its retention and thus effectiveness at a target site. Thus, in one aspect, the disclosed methods and compositions use insertable or implantable devices, such as needles, catheters, stents, seeds, and others disclosed herein, which exploit HGMS principles to locally increase the force on a MDCP at the target site where the MDT article is strategically positioned in the body.

In some examples disclosed herein, a wire or spherical article is positioned at a target (disease) site in a body to locally increase the force on and hence retention of the MDCPs at the site in the presence of an externally applied magnetic field. This external magnetic field magnetically energizes the article, which in turn produces a short-ranged force that positively affects any nearby MDCP due to the local increase in the magnetic field gradient. Thus, the disclosed methods and compositions can be used to treat various disease sites in the body.

In some examples disclosed herein, a wire or spherical article is positioned at a target (disease) site just outside the body to locally increase the force on and hence retention of the MDCPs at the site in the presence of an externally applied magnetic field. This external magnetic field magnetically energized the article, which in turn produces a short-ranged force that positively affects any nearby MDCP due to the local increase in the magnetic field gradient. Thus, the disclosed methods and compostions can be used to treat various disease sites in the body.

In other examples, MDCPs with an encapsulated drug or treatment of choice can be injected into a subject. The focal concentration and release of the encapsulated drug at the target site can be accomplished utilizing a magnetizable article, such as a magnetizable needle, stent, catheter tip, seed, and the like, as are disclosed herein. Magnetizable needles, stents, and catheter tips can be implanted into the target organ or tissue using minimally invasive and conventional techniques such as angioplasty. Magnetizable seeds can be implanted into the target organ or tissue using a relatively noninvasive technique such as through a simple transdermal injection with a syringe.

The methods and compositions disclosed herein can offer better options than other drug targeting approaches because they are universally flexible, tend to be minimally invasive, can be very specific and yet do not rely on complicated biological and chemical interactions.

Disclosed herein, in one aspect, are magnetizable articles that can comprise a magnetizable member. By “magnetizable” is meant that the article can become magnetized (i.e., can exert a localized magnetic field) when placed in an external magnetic field. The disclosed magnetizable articles can also loose their magnetization when the external magnetic field is removed (i.e., the article exerts substantially no localized magnetic field in the absence of the applied external magnetic field). In some specific examples, a suitable magnetizable article can comprise paramagnetic, ferromagnetic, anti-ferromagnetic, ferrimagnetic, or superparamagnetic material. The magnetic force density generated or created by these materials can be in the range from about 1×10⁴ to about 1×10¹⁴ N/m³ when exposed to a magnetic field strength ranging from about 1 to about 8000 kA/m. In some examples, the magnetic force density generated or created by these materials can be from about 1×10⁴ to about 1×10¹⁴, from about 1×10⁵ to about 1×10¹³, from about 1×10⁶ to about 1×10¹², from about 1×10⁷ to about 1×10¹¹, from about 1×10⁸ to about 1×10¹⁰, from about 1×10⁴ to about 1×10⁸, or from about 1×10⁸ to about 1×10⁴ N/m³ when exposed to a magnetic field strength ranging from about 1 to about 8000 kA/m. The magnetic field strength can be from about 1 to about 8000, about 1 to about 800, about 1 to about 80 kA/m, about 100 to about 8000, or about 100 to about 800 kA/m.

As is disclosed and described herein, in one aspect, the article comprises a magnetizable member such as, for example, at least one or a plurality of small paramagnetic, ferromagnetic, anti-ferromagnetic, ferrimagnetic, or superparamagnetic seeds (e.g., ranging in diameter from about 20 nm to 2000 nm) have the innate ability to capture in some cases the far larger magnetic drug carrier particles in capillary and other tissues. Paramagnetic, ferromagnetic, anti-ferromagnetic, ferrimagnetic, or superparamagnetic seeds can be prepared with the most optimal physical and biological properties for magnetic drug targeting using, for example, sonochemical techniques. Further, such paramagnetic, ferromagnetic, anti-ferromagnetic, ferrimagnetic, or superparamagnetic seeds can be implanted and magnetically retained at a target site by using an external magnetic field source. These seeds can significantly enhance the collection of the MDCPs at this site, over that which would be collected simply by using the external magnetic field source alone without the seeds.

These paramagnetic, ferromagnetic, anti-ferromagnetic, ferrimagnetic, or superparamagnetic seeds can be biocompatible in that they are small enough to avoid or delay bioclearance mechanisms of the body, they can magnetically agglomerate at the site thereby facilitating retention of the MDCPs, they can readily de-agglomerate when the magnetic field is removed so that they once again are small enough to be removed from the body by natural means after they have served their purpose. Based on the use of paramagnetic, ferromagnetic, anti-ferromagnetic, ferrimagnetic, or superparamagnetic seeds to enhance the force on and hence retention of magnetic drug carrier particles (MDCPs) (or radioactive particles) at a specified site in the body, such as a tumor, the disclosed magnetic drug targeting article approach can be non-invasive and only require the use of an external magnet, the magnetic seeds, and the MDCPs.

For example, and referring to FIG. 1, a macroscopic view of the affected zone (e.g., a tumor) in the body that needs drug targeting is depicted in FIG. 1a. The bloodstream moves from left to right beginning at an artery that branches into arterioles and then capillaries that irrigate the affected zone. The drugs, which are encapsulated in the MDCPs, enter the zone through the main artery at point A and are magnetically collected somewhere in the capillary system, say at point B, by the superparamagnetic, paramagnetic, ferromagnetic, anti-ferromagnetic, or ferrimagnetic seeds. In some aspects, these seeds can already be placed at this site by first injecting them into the blood stream and then waiting a short time for them to collect at the site under the influence of a magnetic field generated by an external magnet located near the site. One feature is that these strategically positioned magnetic seeds can also be magnetically energized by the externally applied magnetic field.

In one aspect, the seeds are sized and shaped to that they are small enough to allow them to operate effectively in the body while avoiding or delaying the body's natural bioclearance mechanisms, e.g., the immuno-response of the body that removes foreign matter from the circulation system. In one aspect, the seeds are typically less than about 100 nanometers in diameter, which reduces the immuno-response of the body.

In another aspect, the seeds are adapted to allow them to be magnetically directed and fully retained at or near the target site by the magnetic field created by the external magnet. The retention of these seeds at the site can be synergistically facilitated in at least two ways: first, through magnetic agglomeration and second, through magnetic density. Both these attributes can help overcome the hydrodynamic effects of blood flow through the vessel, the primary force that hinders retention of the respective seeds in or at the targeted site. For example, due to the fact that magnetic agglomeration can occur between the seeds once they are exposed to the external magnetic field, clusters or magnetically aligned filaments can form as they become retained. This can aid retention. Also, because they can be comprised of up to 100% magnetic material contained in a very small volume, these seeds, clusters or filaments can be magnetically dense and thus less affected by hydrodynamic forces. Hence, these seeds can more easily retained at the target zone by the external magnetic field compared to the much larger MDCPs without the seeds being present, because the MDCPs typically contain only 2 to 20 vol % magnetic material.

Also, when magnetically energized by the external magnetic field, a seed, cluster or filament formed from the disclosed seeds creates a local magnetic force density that is of sufficient strength to enhance the ability of the external magnet to retain the MDCPs at the targeted site. One will appreciate that, in the absence of a seed, cluster or filament, the intensity and gradients of the magnetic field created by the external magnet alone will, in most cases, not be strong enough (particularly if the external magnet is relatively distant from the site) to retain a significant number of MDCPs. This will allow the MDCPs to escape to other parts of the body before releasing their drug or radiation (as depicted in FIG. 1b.I), possibly causing undesirable side effects.

In one aspect, the seeds readily de-agglomerate when the external magnetic field is removed, which allows the seeds to reenter the blood stream for subsequent removal without causing embolization or necrosis in good tissue. In one aspect, the seeds can be comprised of either a superparamagnetic, ferrimagnetic, or soft ferromagnetic material, which characteristically will lose most, if not all, of its magnetic moment (i.e., remanence) once the magnetic field is removed.

In further aspects, the seeds are sized and shaped for ready removal from the body through naturally means, e.g., through the liver. In one example, superparamagnetic behavior usually appears in seeds that are less than about 50 nm in diameter, which is within the size range to be magnetically strong (especially after agglomeration) and yet still be easily removed by the body.

As previously noted, the development of effective magnetic drug targeting approaches has been hampered by the lack of sufficient retention of the MDCPs at the site due to low magnetic force densities. In contrast, the paramagnetic, ferromagnetic, anti-ferromagnetic, ferrimagnetic, or superparamagnetic seeds disclosed herein, because of the much larger magnetic gradients they create when magnetically induced, are able to fully trap the MDCPs at the zone (as depicted in FIG. 1b.II.a), even if the intensity and gradients of the external magnetic field are small. In use, once the seeds are magnetically positioned as individual particles, clusters or filaments, their relatively large local magnetic field gradients can enhance the collection of the MDCPs. Moreover, since the seeds can be dispersed all around the site (as depicted in FIG. 1b.II.b), the chance of the drug being administered both locally and completely increases dramatically, which also minimizes the occurrence of side effects. Also, once the MDCPs deliver the drug, the external magnet can simply be removed, and the both the MDCPs and the seeds will be carried away by the blood stream for subsequent removal.

The seeds, for example and not meant to be limiting, can be rods or spheres with diameters ranging between 1 and 2000 nm (see FIGS. 13 and 14) that are dispersed along the capillaries of the body or alternatively aligned in the direction of the field to form filaments (as depicted in FIG. 1(c)). In use, it is contemplated that the MDCPs, as they move through the capillaries, meet up with a seed, cluster, or filament and become trapped or attracted thereto (as depicted in FIG. 1(d)). If the local magnetic field and gradients generated by the filament are strong enough, additional MDCPs can also be trapped. As the seeds become saturated with MDCPs, the newly approaching ones can flow past the saturated seeds to meet up with and be retained by other empty seeds positioned further downstream.

In one aspect, a suitable magnetizable seed can be of any shape. For example, a suitable magnetizable seed can have a generally round, oblong, square, rectangular, irregular, cylindrical, spiral, toroidal, ring, spherical, or plate-like shape. Of course, other geometric shapes are contemplated.

In another aspect, a suitable magnetizable seed can be of any size, as long as the seed is biocompatible. For example, a suitable magnetizable seed can have a diameter of from about 1 to about 2000 nanometers, from about 1 to about 1000 nanometers, from about 1 to about 500 nanometers, from about 500 to about 1000 nanometers, or from about 1000 to about 2000 nanometers. In other examples, the seeds can have a diameter of less than about 2000, less than about 1500, less than about 1000, less than about 500, less than about 50, less than about 25, or less than about 15 nanometers. In still another example, a suitable magnetizable seed can have a diameter of about 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1005, 1010, 1015, 1020, 1025, 1030, 1035, 1040, 1045, 1050, 1055, 1060, 1065, 1070, 1075, 1080, 1085, 1090, 1095, 1100, 1105, 1110, 1115, 1120, 1125, 1130, 1135, 1140, 1145, 1150, 1155, 1160, 1165, 1170, 1175, 1180, 1185, 1190, 1195, 1200, 1205, 1210, 1215, 1220, 1225, 1230, 1235, 1240, 1245, 1250, 1255, 1260, 1265, 1270, 1275, 1280, 1285, 1290, 1295, 1300, 1305, 1310, 1315, 1320, 1325, 1330, 1335, 1340, 1345, 1350, 1355, 1360, 1365, 1370, 1375, 1380, 1385, 1390, 1395, 1400, 1405, 1410, 1415, 1420, 1425, 1430, 1435, 1440, 1445, 1450, 1455, 1460, 1465, 1470, 1475, 1480, 1485, 1490, 1495, 1500, 1505, 1510, 1515, 1520, 1525, 1530, 1535, 1540, 1545, 1550, 1555, 1560, 1565, 1570, 1575, 1580, 1585, 1590, 1595, 1600, 1605, 1610, 1615, 1620, 1625, 1630, 1635, 1640, 1645, 1650, 1655, 1660, 1665, 1670, 1675, 1680, 1685, 1690, 1695, 1700, 1705, 1710, 1715, 1720, 1725, 1730, 1735, 1740, 1745, 1750, 1755, 1760, 1765, 1770, 1775, 1780, 1785, 1790, 1795, 1800, 1805, 1810, 1815, 1820, 1825, 1830, 1835, 1840, 1845, 1850, 1855, 1860, 1865, 1870, 1875, 1880, 1885, 1890, 1895, 1900, 1905, 1910, 1915, 1920, 1925, 1930, 1935, 1940, 1945, 1950, 1955, 1960, 1965, 1970, 1975, 1980, 1985, 1990, 1995, or 2000 nanometers, where any of the stated values can form an upper or lower endpoint when appropriate.

Magnetic particles or seeds of various compositions with diameters greater than about 100 nm up to around 2000 nm are readily available or can be synthesized through a variety of conventional techniques that are well known to anyone skilled in the art. The same is not true for magnetic particles or seeds that are less than about 100 nm in diameter down to around 2 nm. Therefore, nanometer-sized solids are the subject of intense and current research owing to their interesting electrical, optical, magnetic, and chemical properties, which often drastically differ from their bulk counterparts. There is a dramatic change in magnetic properties that occurs when the critical length governing magnetic and structural phenomena becomes comparable to the nanoparticle or nano-crystal size. For example, a typical ferromagnetic material exhibits superparamagnetic behavior when its particle size is reduced to about 10 to about 15 nm. Such magnetic nanoparticles are finding applications in magnetic refrigeration, ferrofluids, ultrahigh-density magnetic information storage, contrast enhancement in magnetic resonance imaging, bioprocessing, and magnetic carriers for drug targeting. This phenomenon associated with the size and magnetic properties of magnetic particles is exploited herein to make superparamagnetic nanoparticle seeds for MDT.

It is well known to one skilled in the art that synthesis techniques can provide control over particle or crystallite size, distribution of particle sizes, and interparticle spacing. In the past few years, considerable progress has been made in the controlled synthesis of nanoparticles with sizes ranging from about 2 to about 50 nm. Techniques commonly used for synthesis of nanostructured materials include gas phase methods such as molten metal evaporation and flash vacuum thermal and laser pyrolysis decomposition of volatile organometallics (see Moser, Chim. Ind., 80:191, 1998; Sanchez, et al., J. Mag. Magn. Mater., 365:140-144, 1995; Siegel, Analusis 24:M10, 1996; Siegel, NATO ASI Series, Series E: Applied Sciences 233:509, 1993).

Liquid phase methods use reduction of metal halides with various strong reductants, and colloidal techniques with controlled nucleation (see Moser, Chim. Ind., 80:191, 1998; Hyeon, Chem. Commun., 927-934, 2003). However, sonochemical reactions of volatile organometallics have been added to the vast range of techniques, as a general approach to the synthesis of nanophase materials.

The chemical effects of ultrasound arise from acoustic cavitation—the formation, growth, and implosive collapse of bubbles in a liquid. Violent collapse of bubbles caused by cavitation produces intense localized heating and high pressures. Sonochemical hot spots with effective local temperatures of about 5000 K, local pressures of about 1000 atmospheres, and heating and cooling rates of about 10⁹ K/s are created. The extreme conditions created inside the collapsing bubble are used for the synthesis of unusual materials from volatile organometallic compounds dissolved in the liquid. Ultrasonic reactions normally occur while maintaining a moderate argon flow to facilitate the cavitation process, insure proper mixing of reagents, and elevate the temperature of implosive bubble collapse. Vapors of volatile organometallic precursor penetrate the cavitating bubble, and decompose upon the bubble collapse; the resulting metal atoms agglomerate to form nanostructured materials.

Previous studies have shown that sonochemical synthesis with iron pentacarbonyl, Fe(CO)₅, cobalt tricarbonyl hydrazine, Co(NO)(CO)₃, and similar compounds, yields nanometer-sized magnetic particles, exhibiting superparamagnetic properties (see Cao, et al., J. Mater. Chem., 7:2447, 1997; Grinstaff, et al., Phys. Rev. B, 48:269, 1993; Shafi, et al., J. Appl. Phys., 81:6901, 1997; Shafi, et al., Prop. Complex Inorg. Solids, Prof Int. Alloy Conf., 1^{st}, 169, 1997; Shafi, et al., J. Phys. Chem. B, 101:6409, 1997; Suslick, et al., Mater. Res. Soc. Symp., Proc., 351:443, 1994; Suslick, et al., NATO ASI Ser., Ser. C, 524:291, 1999). Control over the nanoparticle size, as well as over the interparticle interactions, can be achieved by controlling the concentration of reagents, and by introducing surfactants, such as oleic acid, into the reaction vessel. When sonication occurs in the presence of bulky or polymeric surfactants, stable nanophased metal or metal oxide colloids are created (see Shafi, et al., Adv. Mater., 8:769, 1998; Shafi, et al., Thin Solid Films, 318:38, 1998; Suslick, J. Am. Chem. Soc., 118:11960, 1996; Prozorov, et al., Nanostr. Mater., 12:669, 1999). An example is shown in FIG. 13. Surfactants can also be used to stabilize the magnetic nanoparticles in solution.

Various magnetic nanoparticles, including Fe—Co, Fe—Ni, Co—Ni, and Fe—Co—Ni alloys and similar highly magnetic materials have been successfully synthesized, using the sonochemical method. Measurements of the magnetic properties of these nanophased materials have shown high permeability and very small hysteresis values. Preparation of magnetic nanoparticles can be performed via a multi-step process, where synthesis of suitable precursor is followed by sonochemical synthesis and deposition of superparamagnetic particles carried out in the same reaction vessel, while delivering the volatile organometallics via the gas phase. The sonochemical synthesis in a magnetic field produces magnetic nanorods, with a high aspect ratio, as shown in FIG. 14.

Homogeneous sonochemistry in solutions, emulsions and sonochemical sol-gel chemistry can be used for synthesis of metallic and metal oxide nanoparticles. Substitution of conventional ultrasonic bath setup for the direct-immersion geometry can allow for the more effective use of ultrasound and should result in the formation of 3 to 50 nm particles, possibly even other sizes.

Also, articles disclosed herein can be in other forms. For example, the articles can be one or more wires, stents, needles, catheters, catheter tips, coils, meshes, or beads. These can vary in size from the nanometer scale to micro or millimeter scale.

MDCPs are being used today primarily as contrasting agents in MRI; however, they are finding increasing applications as drug targeting devices, which is the subject of this patent. In addition to their use in MRI and MDT, magnetic particles, in general, are finding additional medical applications in separations, immunoassay, and hypertyhermia. (See Shinkai, J. Bioscience and Bioeng., 94:606-613, 2002). This subject has been treated in detail in the open literature (see Momet, et al., J. Mater. Chem. 14:2161-2175, 2004; Tartaj, et al., J. Phys. D:Appl. Phys., 36:R182-R197, 2003; Berry et al., J. Phys. D:Appl. Phys., 36:R198-R206, 2003; Pankhurst, et al., J. Phys. D:Appl. Phys., 36:R167-R181, 2003). Manufacturers and or users of MDCPs include Magforce Nanotechnologies (Berlin, Germany), Nanocet, LLC, Biophan Technologies, Inc. (West Henrietta, N.Y.), and FeRx, Inc. For a general list of magnetic carrier suppliers, which includes medical applications, refer to http://www.magneticmicrosphere.com/supply.htm.

MDCPs can have one or more of the following attributes: they can have a magnetic component and they can have a therapeutic agent. For example, MDCPs in one form can be comprised of a biocompatible polymer shell containing a drug (which can be in liquid form) and magnetic nanoparticles such as magnetite. MDCPs in another form can be comprised of just the magnetic component and used for hyperthermia treatment. MDCPs in yet another form can be comprised of a magnetic component and a radioactive component for radiation therapy. There are many other possible configurations.

In one aspect, the MDCPs that are contemplated for use with the systems and methods disclosed herein can be of any shape or size as long as they do not adversely affect the subject. The MDCP size should be less than about 2 μm in diameter to readily pass through the capillary system and prevent clogging or embolization. For example, the MDCP can comprise a vesicle, polymer, metal, mineral, protein, lipid, carbohydrate, or mixture thereof.

In one aspect, the MDCPs can have a diameter from about 1 to about 2000 nanometers, from about 2 to about 500 nanometers from about 5 to about 150 nanometers, from about 10 to about 100 nanometers, or from about 10 to about 80 nanometers. In one aspect, the MDCPs can have a diameter as disclosed above for the seeds. In still other aspects, the MDCPs can have a diameter of about 0.1, 0.5, 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1000 micrometers, where any of the stated values can form an upper or lower end point where appropriate.

In another aspect, MDCP can comprise magnetite or any magnetic material with a saturation magnetization greater than about 0.1 emu/g, including paramagnetic, ferromagnetic, anti-ferromagnetic, ferrimagnetic, and superparamagnetic materials. For example, the magnetite can be present in an amount of from about 1 to about 98, from about 5 to about 95, from about 10 to about 90, or from about 30 to about 80% by weight, based on the total weight of the particle.

In another aspect, the MDCP can comprise a magnetizable material. For example, the magnetizable material can be present in an amount of from about 1 to about 98, from about 5 to about 95, from about 10 to about 90, or from about 30 to about 80% by weight of the particle. In still other examples, the magnetizable material be present in the MDCP in an amount of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% by weight of the particle, where any of the stated values can form an upper or lower endpoint when appropriate. In one non-limiting example, the magnetizable material can comprise magnetite. In another non-limiting example, the magnetizable material can comprise a mixture or composite of different magnetic materials.

In one aspect, the MDCP can comprise a composition having activity against any disease or disorder. For example, the MDCP can comprise a pharmaceutical composition and/or a radioactive composition. In some specific examples, the MDCP can comprise an agent active against restenosis. Methods for encorporating compositions into a MDCP are known in the art.

Other examples of pharmaceutical compositions that can be used in the MDCP's disclosed herein include, but are not limited to, adrenocortical steroid; adrenocortical suppressant; aldosterone antagonist; amino acids; anabolics; anthelmintic; anti-acne agent; anti-adrenergic; anti-allergic; anti-amebic; anti-androgen; anti-anemic; anti-anginal; anti-arthritic; anti-asthmatic; anti-atherosclerotic; antibacterial; anticholelithic; anticholelithogenic; anticholinergic; anticoagulant; anticoccidal; antidiabetic; antidiarrheal; antidiuretic; antidote; anti-estrogen; antifibrinolytic; antifungal; antiglaucoma agent; antihemophilic; antihemorrhagic; antihistamine; antihyperlipidemia; antihyperlipoproteinemic; antihypertensive; antihypotensive; anti-infective; anti-infective, topical; anti-inflammatory; antikeratinizing agent; antimalarial; antimicrobial; antimitotic; antimycotic, antineoplastic, antineutropenic, antiparasitic; antiperistaltic, antipneumocystic; antiproliferative; antiprostatic hypertrophy; antiprotozoal; antipruritic; antipsoriatic; antirheumatic; antischistosomal; antiseborrheic; antisecretory; antispasmodic; antithrombotic; antitussive; anti-ulcerative; anti-urolithic; antiviral; appetite suppressant; benign prostatic hyperplasia therapy agent; bone resorption inhibitor; bronchodilator; carbonic anhydrase inhibitor; cardiac depressant; cardioprotectant; cardiotonic; cardiovascular agent; choleretic; cholinergic; cholinergic agonist; cholinesterase deactivator; coccidiostat; diagnostic aid; diuretic; ectoparasiticide; enzyme inhibitor; estrogen; fibrinolytic; free oxygen radical scavenger; glucocorticoid; gonad-stimulating principle; hair growth stimulant; hemostatic; hormone; hypocholesterolemic; hypoglycemic; hypolipidemic; hypotensive; immunizing agent; immunomodulator; immunoregulator; immunostimulant; immunosuppressant; impotence therapy adjunct; inhibitor; keratolytic; LHRH agonist; liver disorder treatment, luteolysin; mucolytic; mydriatic; nasal decongestant; neuromuscular blocking agent; non-hormonal sterol derivative; oxytocic; plasminogen activator; platelet activating factor antagonist; platelet aggregation inhibitor; potentiator; progestin; prostaglandin; prostate growth inhibitor; prothyrotropin; pulmonary surface; radioactive agent; regulator; relaxant; repartitioning agent; scabicide; sclerosing agent; selective adenosine A1 antagonist; steroid; suppressant; symptomatic multiple sclerosis; synergist; thyroid hormone; thyroid inhibitor; thyromimetic; amyotrophic lateral sclerosis agents; Paget's disease agents; unstable angina agents; uricosuric; vasoconstrictor; vasodilator; vulnerary; wound healing agent; and xanthine oxidase inhibitor, including mixtures thereof.

As used throughout, administration of any of the MDCPs and/or articles described herein can occur in conjunction with other therapeutic agents. Thus, the MDCPs and/or articles can be administered alone or in combination with one or more therapeutic agents. For example, a subject can be treated with MDCPs and/or articles alone, or in combination with chemotherapeutic agents, antibodies, antibiotics, antivirals, steroidal and non-steroidal anti-inflammatories, conventional immunotherapeutic agents, cytokines, chemokines and/or growth factors. Combinations can be administered either concomitantly (e.g., as an admixture), separately but simultaneously (e.g., via separate intravenous lines into the same subject), or sequentially (e.g., one of the compounds or agents is given first followed by the second). Thus, the term “combination” or “combined” is used to refer to either concomitant, simultaneous, or sequential administration of two or more agents. In one aspect, the MDCPs and/or articles can be combined with other agents such as, for example, Paclitaxel, Taxotere, other taxoid compounds, other anti proliferative agents such as Methotrexate, anthracyclines such as doxorubicin, immunosuppressive agents such as Everolimus and Serolimus, and other rapamycin and rapamycin derivatives.

The MDCPs and/or articles can be administered in a number of ways depending on whether local or systemic treatment is desired, and on the area to be treated. Administration can be topically (including opthamalically, vaginally, rectally, intranasally), orally, by inhalation, or parenterally, for example by intravenous drip, subcutaneous, intraperitoneal or intramuscular injection. The disclosed compositions can be administered intravenously, intraarterialy, intraperitoneally, intramuscularly, subcutaneously, intracavity, transdermally, intratracheal, extracorporeally, or topically (e.g., topical intranasal administration or administration by inhalant). The latter can be effective when a large number of subjects are to be treated simultaneously. Administration of the compositions by inhalant can be through the nose or mouth via delivery by a spraying or droplet mechanism. Delivery can also be directly to any area of the respiratory system (e.g., lungs) via intubation.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein in its entirety for the methods taught.

The compositions can be in solution or in suspension (for example, incorporated into microparticles, liposomes, or cells). These compositions can be targeted to a particular cell type via antibodies, receptors, or receptor ligands. The following references are examples of the use of this technology to target specific proteins to given tissue (Senter et al., Bioconjugate Chem., 2:447-451, 1991; Bagshawe, Br. J. Cancer, 60:275-281, 1989; Bagshawe et al., Br. J. Cancer, 58:700-703, 1988; Senter et al., Bioconjugate Chem., 4:3-9, 1993; Battelli et al., Cancer Immunol. Immunother. 35:421-425, 1992; Pietersz and McKenzie, Immunolog. Reviews, 129:57-80, 1992; Roffler et al., Biochem. Pharmacol., 42:2062-2065, 1991). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis have been reviewed (see Brown and Greene, DNA and Cell Biology 10:6, 399-409, 1991).

For additional discussion of suitable formulations and various routes of administration of therapeutic compounds, see, e.g., Remington: The Science and Practice of pharmacy (19th ed.) ed. A. R. Gennaro, Mack Publishing Company, Easton, Pa. 1995.

As disclosed herein, the MDCPs and/or articles are administered to a subject in an effective amount. By “effective amount” is meant a therapeutic amount needed to achieve the desired result or results, e.g., treating or preventing restenosis or cancer. The exact amount of the compositions required will vary from subject to subject, depending on the species, age, weight and general condition of the subject, the severity of the disorder being treated, its mode of administration and the like. Thus, it is not possible to specify an exact amount for every composition. However, an appropriate amount can be determined by one of ordinary skill in the art using only routine experimentation given the teachings herein.

The MDCPs and/or articles can be used therapeutically in combination with a pharmaceutically acceptable carrier. Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

In one aspect, any of the MDCPs and/or articles described herein can be combined with at least one pharmaceutically-acceptable carrier to produce a pharmaceutical composition. The pharmaceutical compositions can be prepared using techniques known in the art. In one aspect, the composition is prepared by admixing the ribonucleotide reductase inhibitor having with a pharmaceutically-acceptable carrier. The term “admixing” is defined as mixing the two components together so that there is no chemical reaction or physical interaction. The term “admixing” also includes the chemical reaction or physical interaction between the ribonucleotide reductase inhibitor and the pharmaceutically-acceptable carrier.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, anti-inflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

In addition to the use of the disclosed magnetizable articles as magnetic seeds, they can alternatively be used to aggressively treat cancerous tumors. For example, under the influence of an external magnetic field, magnetic particles can be used to force localized embolization or necrosis of affected capillaries, thereby starving a tumor of blood. These magnetic particles can also be used as a hyperthermia agent under the influence of an alternating magnetic (AC) field, thereby killing the tumor through localized heating. This is made possible again through the use of an external magnetic field source for retaining the magnetic particles essentially at the targeted site therein the body.

### EXAMPLES

The following examples are put forth so as to provide those of ordinary skill in the art with a complete disclosure and description of how the compounds, compositions, systems, articles, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g. MDCP concentrations and their composition, type of magnetizable article and its composition, and type of magnetic field generator that can be used to optimize the performance of the MDT system or device. Only reasonable and routine experimentation will be required to optimize such process conditions.

### Example 1

A schematic of the control volume (CV) utilized for modeling the capture of magnetic drug carrier particles (MDCPs) is shown in FIG. 2. The fluid dynamics are represented by the equation of continuity and the Navier-Stokes Equations for a Newtonian fluid Bird et al., Transport Phenomena 2d Ed., Wiley & Sons, New York, 2002):

$$\begin{matrix}∇ \cdot \underline{v} = 0 & ( 1 ) \\ ρ [ \frac{\partial \underline{v}}{\partial t} + ( ∇ \underline{v} ) \cdot \underline{v} ] = - ∇ P + ∇ \cdot η ( ∇ \underline{v} + {( ∇ \underline{v} )}^{T} ) & ( 2 )\end{matrix}$$

where ν is the velocity, ρ the blood density, P the pressure and η is the blood viscosity. “No slip” boundary conditions are used at interfaces, the pressure is defined as 101325 N/m at the external carotid artery (ECA) and internal carotid artery (ICA) exits. Additional assumptions associated with this model include isothermal behavior, incompressible Newtonian fluid, rigid walls, and single-phase flow. The initial velocity entering the common carotid artery is described by a Fourier series, approximating a real pulsatile flow during a cardiac cycle, as shown in FIG. 2 and described by:

$${v}_{avg} ( t ) = {\sum }_{n = 0}^{15} {a}_{n} cos ( nt ω + {θ}_{n} ) \qquad ( 3 )$$

For αₙ, and θ values see Table 1. The entering flow is modified to specify a parabolic profile, as:

$${v}_{x} {\vert}_{x = 0} = 1.5 {v}_{avg} ( t ) ( 1 - {( \frac{y}{{R}_{CCA}} )}^{2} ) \qquad ( 4 )$$

where ν_{avg} (t) is the average inlet velocity from and described by Eq. (3), R_{CCA} is the radius of the CCA at the entrance, y is the position from the CCA center.

The magnetic field within a specified CV is described by the Maxwell equation:
∇²φ=0 (5)
where φ is the magnetic potential. The regions inside the wire and outside the ferromagnetic wire have dissimilar properties, which obligates to define two magnetic potentials for each region as:
∇²φ₁=0 (6)
∇²φ₂=0 (7)

The magnetic fluxes (B) in the space can then be calculated as:
Bₒ=Hₒ(Hₘ−∇φ₁) (8)
Bᵢ=μₒ((M_{c}+Hₘ)−∇φ₂) (9)
where M_{c} is the induced magnetization of the wire parallel to the applied field, μₒ is the magnetic permeability of free space and Hₘ is the applied field, which can be either homogeneous:
H_{m,x}=Hₒ cos(γ) (10.a)
H_{m,y}=Hₒ sin(γ) (10.b)
or generated by a magnet of magnetization Mₘ, the field of which is assumed to be equal to that of infinitely long cylinder of radius Rₘ perpendicular to the plane of FIG. 1. In cylindrical coordinates, this field is given by

$$\begin{matrix}{H}_{m , r} = 0.5 {{M}_{m} ( \frac{{R}_{m}}{r} )}^{2} cos ( θ - γ ) & ( 11. a ) \\ {H}_{m , θ} = 0.5 {{M}_{m} ( \frac{{R}_{m}}{r} )}^{2} sin ( θ - γ ) & ( 11. b )\end{matrix}$$

where r is the distance to the center of the magnet, θ the angle formed between a parallel line in the x direction and the point of evaluation. When the applied field is homogeneous, γ is the angle between the direction of applied field and the parallel line in the x-direction. When the applied field is generated by the magnet, γ is the angle between the direction of internal field within the magnet and the parallel line in the x-direction. These equations are then transformed into Cartesian coordinates, for further evaluation. The total field outside of the magnet is defined as:
H_{field}=Hₘ−∇φ (12)

For a MDCP in a fluid with volume Vₚ, radius Rₚ, porosity εₚ and ferromagnetic weight content ωₚ, and submerged in a magnetic environment, the forces that affect the particle are:

$${\underline{F}}_{d} + {\underline{F}}_{m} = {m}_{p} * \frac{ⅆ {\underline{v}}_{p}}{ⅆ t} \qquad ( 13 )$$

where F_{d} is the drag force, Fₘ the magnetic force, mₚ the particle mass, νₚ the particle velocity and Mₚ is the particle magnetization. It is assumed that Mₚ has the same direction as H_{field}.

From Eq. (13), and neglecting inertial forces, the particle velocity can be expressed in explicit form

$${\underline{v}}_{p} = \underline{v} + {V}_{m} \frac{{R}_{w}}{{M}_{c} {H}_{field}} ∇ ( {\underline{H}}_{field} \cdot {\underline{H}}_{field} ) \qquad ( 14 )$$

where Vₘ is the magnetic velocity and is defined as:

$${V}_{m} = \frac{2}{9} \frac{{R}_{p}^{2}}{{R}_{w}} \frac{{μ}_{o}}{{η}_{B}} ( 1 - {ɛ}_{p} ) {ω}_{fm , p} {M}_{fm , p} {M}_{c} \qquad ( 15 )$$

with ωₚ being the volumetric fraction of magnetite and it is related to the weight content x_{fm} through:

$${ϖ}_{fm , p} = \frac{{x}_{fm}}{{x}_{fm} + ( 1 - {x}_{fm} ) \frac{{ρ}_{fm}}{{ρ}_{pol}}} \qquad ( 16 )$$

Finally, the particle trajectories are obtained according to streamline functions:

$$\frac{\partial ψ}{\partial y} = - {v}_{p , x} ; \frac{\partial ψ}{\partial x} = {v}_{p , y} \qquad ( 17 )$$

This model was used to study the targeting of magnetic particles at a specific zone at the CCA-ICA split. Table 1 shows the parameters used in the model. In FIG. 4, a simulation carried out in the FEMLAB platform is shown. The magnet is predefined and represented by Equations 8 and 9. The fluid dynamics and magnetic equations were solved independently. The streamlines show particle trajectories and were calculated using Equations 14 and 15. The localized collection zone is specified by an area surrounding the wire and is arbitrary chosen to be 3 times the size of the wire. The background represents the magnetic field gradient and a higher magnetic field at the area immediately surrounding the wire is observed.

Considering the fluid aspects, when a realistic periodic pulsatile is used in the analysis of particle collection, flow changes can be observed at different times during one pulse. From FIG. 3, the flow starts as a flat profile (velocity about 0.2 m/s), the velocity accelerates up to a maximum (systolic point). This velocity decelerates (end of systolic point) and small variations of the flow are seen until the flow is stabilized again and another pulse is repeated. When this velocity profiles are used the complexity in the carotid artery is observed. In FIG. 5 flow streamlines are shown for the (a) flat profile, (b) high systolic point, and (c) the end of systolic point.

In FIG. 5(a) the flow streamlines appear to be continuous through the artery, except at the ICA-CCA split zone, where flow helices are seen. These helices are related to secondary flows and flow separation zones associated with the carotid sinus (Bharvadaj, et al., J. Biomechanics 15(5):363-378, 1982; Marshall, et al., J. Biomechanics 37:579-687, 2004; Botnar, et al., J. Biomechanics 33:137-144, 2000).

In FIG. 5(b) the zone of the complex flow in the ICA is characterized by helices and associated with secondary flows. This area increases and occupies the zone between the CCA-ICA split into the sinus. This increase is due to the increase in velocity as observed by Bharadvaj, who observed that the region of flow separation increased with Reynolds number, thus an increase with fluid velocity (Bharvadaj, et al., J. Biomechanics 15(5):363-378, 1982). This observation also explains the smaller zone found at FIG. 5(a).

FIG. 5(c) shows two areas of complex flow along the ICA. The first one is seen at the sinus, and no helixes are observed at the CCA-ICA split. Other smaller helices are seen at the lower wall of the ICA. These complex flows observed at the carotid artery are due to the complex geometry of the artery.

Three MDT systems are compared: 1) the use of a permanent magnet (Mₘ=1,200 kA/m, Rₘ=6.2 cm) combined with a wire (c_{w}=1000, M_{w,s}=1,650 kA/m, R_{w}=1.55 mm), 2) the use of a permanent magnet alone, and 3) the use of a homogenous magnetic field (Hₒ=538 kA/m) combined with a wire. This study verifies the feasibility of collecting particles at the carotid artery bifurcation. The main focus is the collection of particles at a specified targeted site or zone rather than at any position in the vessel. The two main aspects presented are the effects of particle (agglomerated) size and magnetite content in the MDCPs. The performance of the magnetic drug targeted system is described by the collection efficiency (CE). Particle collection is calculated by the percentage of MDCPs that enter the CCA and are collected at the targeted zone.

In FIG. 6 FEMLAB simulations are shown for three different times during one cycle. FIGS. 6(a-c) show the simulation for the case of a wire and external magnet. FIGS. 6(d-e) show the simulations for an external magnet only. When comparing FIGS. 6(a) and (d), the magnet and wire show an increase in the retention of the MDCP versus the magnet alone. In this case the velocity at the CCA entrance is at its lowest point (See FIG. 3). In FIGS. 6(b) and (e), the velocity is at the systolic point, and the collection and collection difference between the two cases is reduced. In FIGS. 6(c) and (f), the retention difference is again increased in favor of the stainless steel wire and magnet. The limiting case is that shown in FIGS. 6(b) and (e), where the velocity is at its maximum during the systolic point. While this is the limiting case, higher collection of particles should be attainable for time fractions during the cardiac cycle.

FIG. 7 shows the collection efficiency as a function of the particle size and magnetic material content for the case of a wire and magnet, magnet alone and a wire in a homogenous field. The velocity at high systolic point was used since it represents the limiting case in the collection. In FIG. 7(a) the collection efficiency (CE) is plotted versus the particle radius (Rₚ) at two different magnetite contents (x_{fm}=0.5, x_{fm}=0.8). In FIG. 7(b) the collection efficiency (CE) is plotted versus the magnetite content for particle radius of 20 μm and 50 μm.

Particle collection increases with both particle size and magnetic material content. The magnetic force is proportional to the magnetic field and the magnetic field gradient, but it also depends in the MDCP properties. At higher particle sizes, the magnetic force increases, increasing collection. The same is true for the magnetic material content of the MDCP. Collections of 100% are possible for large particle sizes and high magnetic material, such as, for example, magnetite content. Collection of 30-60% are possible for particles with the radius of about 20 to about 40 μm when the magnetic material content is about 0.8, and about 30 to about 50 μm when the magnetic material content is about 0.5. Collection is higher for the magnet and wire compared with the other two cases. When compared with the magnet alone, the collection is higher until a maximum collection is reached. The homogenous field has the lowest collection of the three.

In FIG. 7(a) it can be seen that the magnetic material content appears to shift the collection curve to the right as the magnetic material content decreases. This observation is due to the effect that the magnetic material has on the particle properties. On the other hand, FIG. 7(b) shows that the collection curve shifts downward as particle size decreases. The particle size and magnetic material content effects are due to the relation of the magnetic force with the MDCPs properties.

Qualitatively, it appears that the particle collection is higher for the wire and magnet when compared to the other cases. From these plots it can be seen that the magnet and wire combination always have a higher collection than any of the other two cases understudy. This observation is due to the localized field, seen in FIG. 4, that increases the magnetic field around the wire, thus increasing the magnetic field gradient and consequently the magnetic force.

The exemplified model shows targeted drug delivery system to the carotid bifurcation area. This system can, in one example, be used in the treatment of restenosis, after surgical treatments like endarterectomy or angioplasty. The system comprises an article, such as, for example, a wire, needle or the like, which is located just outside the artery wall, close to the sinus at the carotid bifurcation. In this case, the wire can also be located just outside the body adjacent to the skin, since the carotid artery is located so close to the surface of the skin. A magnetic field generated by a permanent magnet, electromagnet, or superconducting magnet can then be used to generate a magnetic force about the article, which comprises a magnetizable member, to collect the delivery particles thereon.

The study indicates that particles agglomerate to create large particle clusters that are collected at a specified target site. Second, that these particles will de-agglomerate when the magnetic field is removed, or at a short distance from it, thus permitting the flow of these particles through the capillaries.

The study concluded that increasing particle size and magnetic material content increased particle retention for all the cases studied and the difference in capture by the magnet and wire also increased. The results show that the magnetic field combined with a wire increased the capture of the particles at the targeted zone, increasing particle collection, and thus the efficiency of a magnetic targeted drug delivery system of the type described herein.

TABLE 1
Values and ranges of the physical parameters used in the
MDT system model and parametric study.

| | | |
|---|---|---|
| Properties | Units | Value(s) |

Blood

| | | |
|---|---|---|
| ρ_{b} | kg/m³ | 995 |
| η_{b} | kg/(m s) | 3.0 × 10⁻³ |
| χ_{b} | SI | 0 |

Drug Carrier

| | | |
|---|---|---|
| material | — | Polymer, Fe, Fe₃O₄ |
| ρ_{pol,p} | kg m⁻³ | 950 |
| χ_{,pol,p} | SI | 0 |
| ρ_{fmp} | kg m⁻³ | 5180 |
| M_{p,s} | kA/m | 480 |
| χ,_{fmp} | SI | 100 |
| X_{fm,p} | % | 20-100 |
| Rₚ | μm | 5-150 |
| εₚ | — | 0.4 |

Wire Material (SS)

| | | |
|---|---|---|
| R_{w} | mm | 1.55 |
| M_{w,s} | kA/m | 1650 |
| χ,_{w} | SI | 100 |
| Magnet material | — | Nd₂Fe₁₄B |
| Form | — | Cylindrical |
| Rₘ | cm | 6.2 |
| Mₘ | kA/m | 1200 |
| β | — | π/4 |

Homogenous Magnetic Field

| | | |
|---|---|---|
| β | — | π/4 |
| Hₒ | A/m | 537780 |

### Example 2

FIG. 8 shows a schematic that depicts an in-vitro experimental setup that can be used to demonstrate the concept of magnetic seeding. The experimental setup comprises a glass tube (1 to 4 mm in diameter) with a 1 cm section containing a fritted glass plug with of 10 μm pores that represents a capillary network; a NdFeB permanent magnet (Magnet Sales and Manufacturing Inc., Culver City, Calif.) of various shapes that is adjacent to the fritted glass section; a flexible tube with two points of injection connected upstream to the glass tubing; two syringes (1 ml and 50 ml) to supply suspensions containing the surrogate MDCPs (Bangs Laboratories, Inc., Fisher, Ind.) and the magnetic seeds (prepared by USC, see example 6 or Nanomat, Inc., North Huntington, Pa.), respectively; and a syringe pump (Cole Palmer 74900, Cole Palmer, Vernon Hills, Ill.) to control the flow of the 50 ml syringe (Hamilton Gastight #1050 Luer Lock, Hamilton, UK).

The magnetic seed articles, which can be dispersed in an aqueous suspension between 0.1 and 0.5 ml, can be injected first using the 1 ml syringe. The magnetic seed articles comprised particles of cylindrical or spherical shape of sizes varying from 20 to 200 nm made of, for example, a superparamagnetic alloy or oxide that can be suspended in solution with the aid of a surfactant (e.g., oleic acid). While the magnetic seed articles are injected, the syringe pump with the 50 ml syringe will supply distilled water at a rate such that the velocity of the solution through the fritted glass pores is about 0.1 cm/s, which is typical of blood flow through capillaries. The permanent magnet, separated from the fritted glass section a distance that is defined by x, magnetically captures the ferromagnetic seeds at the fritted glass. The degree of dispersion of the ferromagnetic seeds throughout the fritted glass is controlled by the concentration of seeds in the suspensions, the shape of the permanent magnet and the distance x, the last two defining the intensity and patterns of the magnetic field at the fritted glass. At this point, the role of surfactant of keeping the seeds apart ceases to be significant and the surfactant is washed away without affecting the role of the seeds. Once the seeds are collected in place, the magnet that is originally located at a distance x=x₁ is brought closer to the fritted glass to a new distance x=x₂. The syringe pump with the 50 ml syringe is then used to supply a suspension of the magnetic particles at the same velocity of 0.1 cm/s. These particles, which represent the MDCPs, are made of a combination of magnetite (between about 5 and about 40 wt %) and polystyrene, with a mean particle diameter from about 0.5 to about 2.5 micrometers. By means of the induced magnetic field of the captured magnetic seeds in the fritted glass and possibly due, in part, to the field of the permanent magnet, the magnetic particles are captured.

A turbidimeter (HACH 2100N, Hach Co., Loveland, Colo.) can be used to determine the concentration of particles of the effluents that are collected in the recipients placed at the end of the glass tubing. FIG. 9(a) shows a calibration curve using the turbidimeter for 2.35 micrometer particles containing 20 wt % magnetite. The magnetic behavior of a dry sample of this same material is also shown in FIG. 9(b). The capture efficiency of the fritted glass with magnetized seeds is then calculated by contrasting these concentrations with that of the original solution in the 50 ml syringe.

There are three magnetic sources that can be used in these experiments; all of them consisting of NdFeB permanent magnets (Magnet Sales and Manufacturing Inc.). The first two sources comprised individual 0.6T magnets; one being a “donut” like magnet and the other being “cube” magnet. The donut magnet has an ID of 12 mm, an OD of 53 mm with a thickness of 15 mm, with the field parallel to the bore. The cube magnet is 50×50×25 mm, with the field perpendicular to the 50×50 mm faces. The third magnetic field source is a magnetic assembly that comprised two, 0.8 T 30×40×50 magnets bolted into a KURT D675 vise that is also used to separate the magnets and vary the field in the space between them. The magnetic field is measured using a F. W. Bell Gauss/Tesla Meter Model 4048.

### Example 3

In this example, magnetic seeds, either purchased from Nanomat, Inc. or prepared as demonstrated in Example 6 below, can be used. The variables included the distance x₁ (for example, varying from about 0 to about 10 cm), the type of magnet (for example, using a cube, “donut,” or dual block magnets), the flow velocity (for example, from about 0.1 to about 0.3 cm/s), the concentration of seeds in the doping solution, and the dimensions of the fritted glass. . Other variables are the role of distance X₂ (0 to 10 cm), the degree of collection of seeds, the concentration of the MDCPs, and the role of each of the following elements when systematically removed from the MDT system, i.e., a) without the seeds, b) without the magnet, or c) without the fritted glass.

Magnetic agglomeration plays a role in both the collection of the seeds and the subsequent collection of the MDCPs by the seeds. Therefore, the concentrations of both the magnetic seeds and the MDCPs are parameters to consider, because their respective concentrations can have a direct impact on their ability to magnetically agglomerate in the presence of the magnet field. For example, the syringes are used in a batch mode to represent high concentrations of slugs of particles being injected in a short time, or in a continuous mode to represent a more evenly dispersed administration of particles injected over a longer period of time. In either case, the same amount of particles is included in the total injected amount to make a fair comparison of the results.

FIG. 10 shows results of magnetic capture experimental studies using a system similar to that depicted in FIG. 8 and magnetic particles (Rₚ=1.165 μm, 20 wt % magnetite, Bangs Laboratories Inc.). The only difference between the two systems is that the system in FIG. 10 studied the behavior of a 1 cm long, home made, ferromagnetic stent inside a 1 mm glass tube instead of the fritted glass-magnetic seed system. The technique was quite effective, with trends that are devoid of noise. It is clear that the observed collection is due to a magnetic effect. For example, the role that the ferromagnetic wire or stent or surrogate seed plays on improving the collection of the magnetic particles is revealed very clearly. Because it is attached to the glass section containing the stent (x=0), the magnet alone also exhibits some collection ability. However, little or no collection was observed with the stent in place without the external permanent magnet to magnetically energize it. Because of the much larger concentrations that result by adding the same amount of magnetic particles batch-wise in 0.1 ml doses, compared to adding them continuously in a 50 ml solution, magnetic agglomeration is facilitated of the MDCPs and hence larger captures were observed.

### Example 4

FIG. 1(e) shows a simple schematic of the control volume (CV) that can be used to create a model of the system disclosed herein. It comprises a horizontal cylinder of radius r_{C} and length L representing a capillary. A stack of N_{nd} spherical seeds of radius r_{nd} is resting at the bottom of the capillary aligned either with the field (as depicted in 1.e.I.a) or along the axial direction of the capillary (as depicted in FIG. 1.e.I.b), with the first seed located at distance L_{T} from the upstream end of the cylinder. If aligned in the direction of the capillary, they can be separated by an inter-particle distance h. The blood, with viscosity μ_{B} and density ρ_{B}, can enter with a mean velocity defined be a parabolic profile at the upstream end.

The pressure and velocity profiles in this CV were determined numerically by solving Navier-Stokes and continuity equations. The description of the magnetic field in the CV was obtained by solving Maxwell equations for conservative magnetic fields, i.e., with the Laplacian of the magnetic potentials being set equal to zero. For this purpose, the CV, defined as a cubic box with sides twice the size of the capillary length, can symmetrically contain the capillary. Each of the faces of the box was far enough from the seeds to assume that the magnetic potential is zero along the boundaries of the box. Magnetically speaking, the space within the box was divided into two regions: one which is magnetic and consisting of the volume of the seeds (present as individual seeds, clusters, or filaments), and one which is non-magnetic and comprising the volume of the rest of the space within the box, including the blood (which is only weakly paramagnetic).

The goal was to predict the trajectories of the MDCPs as they travel through the CV and are influenced by both hydrodynamic and magnetic forces; and then to determine the conditions that lead to magnetic retention of the MDCP by the seeds, as readily indicated by the paths taken by these trajectories. In this way, the feasibility or performance of a MDT system as disclosed herein is defined in terms of the fraction of MDCPs that enter the CV and end up being magnetically retained at the seed, cluster, or filament. Thus, three different sets of differential equations that describe different physical aspects of the dynamics occurring within the CV were formulated and solved sequentially. The simultaneous solution to the first set of equations that describe the x, y, and z components of the blood velocity and the spatial variation of the blood pressure in the CV was obtained by solving four equations, namely the continuity and three Navier-Stokes equations for 3-D systems. The simultaneous solution to the second set of equations that describe the magnetic potential of the two magnetically different regions in the CV was obtained by solving the Maxwell continuity equation for conservative magnetic systems. Hence, the first part of the model consists of three equations, ie., the dimensionless forms of the mass continuity and Navier-Stokes equations (which accounts for three equations) that are solved for four unknowns, namely the three dimensionless components of the blood velocity (i.e., ν_{B,x}, ν_{B,y} and ν_{B,z}) and the dimensionless blood pressure (i.e., π). The second part of the model comprises the two Laplacian equations that are solved for two unknowns, i.e., φ₁ and φ₂. These six equations were solved numerically for the six unknowns using FEMLAB. Finally, in the third part of the model, the information obtained from the solutions to the first two sets of equations, namely ν_{B,x}, ν_{B,y}, ν_{B,z} and φ₂, was used as input to a system of equations that describe a force balance over one MDCP that includes only the magnetic and hydrodynamic forces. This allows for an explicit formulation of the components of the MDCP velocities to be obtained in terms all the system variables and parameters. These velocities were then used to map the trajectories of the MDCPs under the influence of the magnetic and hydrodynamic forces via analysis of their corresponding streamline function. A quantitative description of this three-part model, including all the equations is given in Example 1 and elsewhere (see Ritter, et al., J. Magn. Magn. Mater. 280:184-201, 2004; Chen, et al., J. Magn. Magn. Mater., 284:181-194, 2004; Aviles, et al., J. Magn. Magn. Mater. 293:605-615, 2004; Chen, et al., J. Magn. Magn. Mater., 293:616-632, 2005).

In the third part of the model, the MDCPs were treated as freely moving point masses in the CV fluid, i.e., in the blood; hence, they do not have to satisfy the incompressible fluid form of the continuity equation. In other words, the concentration of the MDCPs was not necessarily constant and allowed to vary within the CV. Other forces not considered in this analysis were inertial, lift, wall effects, gravitational, buoyant, drag forces in non-spherical agglomerated particles, and inter-particle magnetic forces between MDCPs.

### Example 5

Seed anchoring and filament formation were studied. Variables that were considered included the size (40 to 100 nm), concentration and saturation induced magnetization (400 to 1500 kA/m) of the seed, the blood velocity (0.1 to 0.3 cm/s), the capillary diameter (2.5 to 4 μm), the distance x (0 to 10 cm) from the external permanent magnet of given magnetization, size and shape. In the MDCP capture study, for a given seed, or filament or cluster thereof, the variables of interest included blood velocity (0.1 to 0.3 cm/s), capillary diameter (2.5 to 4 μm), magnetic field strength due of the external magnet, size (400 to 2000 nm) of the MDCP, the saturation magnetization (400 to 1500 kA/m) and content (5 to 50 wt %) of the ferromagnetic material in the MDCP, number of MDCPs and whether they formed filaments in the direction of the field or align in the axial direction of the capillary separated with an interparticle distance h (10 to 100 times the nano-docker radius). In all simulations, the blood viscosity ν_{B} and blood density ρ_{B} was typical of that in capillaries (i.e., μ_{B}˜3 μ_{water} and ρ_{B}˜ρ_{water}).

FIG. 11 shows preliminary collection efficiency results (FEMLAB) of six different magnetic seed systems in a capillary using the 2-D streamline analysis approach based on the procedure described above. In this approach the walls of the capillary (R_{c}=4 μm) and the magnetic seeds (R_{nd}=20 nm, Mₛₐₜ=1350 kA/m) are represented by two planes and wires, respectively, all being perpendicular to the plane of the figure. The magnetic field (1.5 T) lies in the plane of the figure and is perpendicular to the blood flow, which enters the capillary with a parabolic profile and mean velocity of 0.1 cm/s moving from left to right. By assuming that the MDCPs (Rₚ=1 μm, 40 wt % magnetite (ρ=5.2 g/cm³, Mₛₐₜ=480 kA/m)) enter the capillary evenly distributed, and that the origin is placed at the mid point of the capillary, the collection efficiency (CE) of this MDT system was defined as CE=[y*+(R_{c}−Rₚ)]/[2(R_{c}−Rₚ)], where excluded volume of the magnetic particles has been considered. The value y* represents the location of the farthest streamline from the bottom end of the capillary that is captured by the magnetic seeds. If y* was such that CE becomes negative, then CE must be zero. FIG. 11(a) shows the CE of a single seed, showing a value of about 6% despite the fact that the seed is 200 times smaller than the capillary. FIGS. 11(b-d) show the effect of the interparticle separation h on CE in a 10 magnetic seed system aligned along the axial direction of the capillary. Notice the additive effect on the CE when the seeds are closer to each other. The calculated CEs were 10.24, 15.11, and 20.64%, respectively, for h=50, 25 and 10 times the seed radius. FIGS. 11(b), (e), and (f) show the effect of the number of seeds aligned in the axial direction of the capillary for an interparticle distance h equal to 10 times the seed radius. Adding particles also has an additive effect on the CE. For example, CEs of 17.30, 20.64, and 24.94% are obtained for 5, 10, and 20 seeds, respectively. In summary, the results show that a plurality of these seeds distributed in a large capillary system can lead to the total collection of the MDCPs. A magnified view of the results observed in FIG. 11(e) is shown in FIG. 12 and clearly show the MDCPs “collecting” around the seeds.

### Example 6

The direct sonochemical decomposition of volatile organometallics was used for the synthesis of superparamagnetic nanoparticles within the 5 to 100 nm range. Magnetic fluids containing nanostructured iron oxide, Fe₂O₃, as well as cobalt and copper ferrites CoFe₂O₄ and CuFe₂O₄ were prepared by sonochemical irradiation of alcohol solutions of iron pentacarbonyl in the presence of bulky stabilizers (oleic acid, or trioctylphosphine oxide (TOPO)), and cobalt- and copper 2-ethylhexanoates. Synthesis from the decane solutions containing 10 μmol to 10 mmol of Fe(CO)₅, and stoichiometric (5 μmol to 5 mmol) amounts of cobalt- and copper 2-ethylhexanoates were also carried out. Control over the particle size was achieved by varying the concentration of the volatile organometallic precursors, and by varying the reaction times and temperatures. Additionally, the rates of nucleation and growth of the as-formed nanoparticles was controlled by the molar ratio of concentrations of organometallic precursor to oleic acid (stabilizer). Two ratios of molar concentrations of [Fe(CO)₅]:[stabilizer] were studied to determine the effect on particle size, i.e., ratios of molar concentrations 0.1:1 and 1:5 were be used to obtain nanoparticles in the 100 nm and 5 nm range, respectively.

The ultrasonic spray pyrolysis method, enabling formation of the finest mists known to date, was used for synthesis of monodispersed nanoparticles with desired particle size. In ultrasonic spray pyrolysis synthesis, a precursor solution was nebulized with a high-frequency ultrasound generator into a heated column-type furnace, where small droplets coalescence in a heated gas to produce a nanostructured material. The resulting nanoparticles were collected in a liquid trap and then precipitated at a later stage of synthesis. Droplet size in this case was largely determined by the frequency of ultrasound used (20 kHz-1 mHz). Chemical composition of the yielded nanoparticles was controlled by simultaneous nebulization of several precursor solutions into a single tube furnace. To prevent particle agglomeration, the salt-assisted spray pyrolysis method was explored to achieve even smaller nanoparticles. The incorporation of simple salts, e.g., KCl, NaCl, into the precursor solution, will cause the final oxide product to be encapsulated in a salt particle. Each droplet generated numerous smaller particles and hence smaller nanoparticles. The ultrasonic spray pyrolysis synthesis of iron oxide, cobalt- and cupper ferrite nanoparticles from 10 mmol solution of corresponding nitrates in the presence of variable amounts of KCl or NaCl was attempted. Subsequent dissolution of the salt matrix in the presence of sodium citrate as a stabilizer, allowed the nanoparticles to be harvested while preventing their agglomeration.

### Example 7

The traditional MDT approach involves the direct and noninvasive application of a permanent magnet to the skin located directly over the affected zone in the body (Ramchand, et al., J. Pure App. Phy. 39(10):683-686, 2001; Babincova, et al., Z. Naturforsch. C. 55(3-4):278-281, 2000; Alexiou, et al., Cancer Res. 60:6641-6648, 2000; Goodwin, et al., J. Magn. Magn. Mater. 194:132-139, 1999; Rudge, et al., J. Control. Release 74:335-340, 2001; Viroonchatapan, et al., Life Sci. 58(24):2251-2261, 1996). The magnet creates a magnetic field with intensity H and gradients ∇H that are supposed to be strong enough to retain MDCPs as they pass through a diseased region located at some distance below the skin. Since, the force exerted on a MDCP (Fₘ) is directly proportional to both the strength (H) and the gradient of the magnetic field (∇H) (Gerber, Magnetic Separation, in: Gerber, et al., (Eds.), Applied Magnetism, NATO ASI Series, Series E: Applied Sciences, Vol. 253, Kluwer Academic Publishers, Dordrecht, 1994, p. 165),i.e.,
Fₘ∝H·∇H. (18)
one way to locally increase the gradient of the magnetic field is to place a ferromagnetic wire in the region of the magnetic field. The large magnetic field gradients that form locally around the wire are due to it becoming energized by the applied magnetic field, which in turn creates its own magnetic field locally around itself. The higher the curvature of this wire (i.e., the smaller the diameter), the larger the gradient of the magnetic field, the greater the force exerted on the MDCPs.

The schematic in FIG. 15 shows that a MIS wire of radius R_{w} is placed perpendicular to the plane of the figure and facing the blood that is flowing across the wire from left to right at velocity u_{b} (also in the plane of the figure). This blood transports the MDCPs of radius Rₚ to the wire for possible capture. The applied magnetic field Hₒ also lies in the plane of the figure and points in the direction defined by angle θ. The performance of the wire is evaluated in terms of its capture cross-section y_{w}, which represents the maximum perpendicular distance that a MDCP can be from the flow streamline that passes through the center of the wire and still be retained. A correlation developed by Ebner and Ritter (Ebner and Ritter, AIChE Journal 47:303, 2001) is utilized here to evaluate the capture cross-section y_{w} under the transversal configuration, i.e., when the magnetic field and the blood flow are aligned perpendicular to each other (θ=π/2).

This correlation assumes that the wire is clean and cylindrical in shape, the blood moving past the wire is governed by the potential flow regime, the blood and the MDCPs are not affected by walls, and the only forces acting on the MDCPs are magnetic and hydrodynamic. All other forces, such as inertial, gravity and Brownian are considered to be unimportant, as expected for liquid systems like blood and the range of the MDCP sizes studied here (Gerber, Magnetic Separation, in: Gerber, et al., (Eds.), Applied Magnetism, NATO ASI Series, Series E: Applied Sciences, Vol. 253, Kluwer Academic Publishers, Dordrecht, 1994, p. 165; Ebner and Ritter, AIChE Journal 47:303, 2001; Cummings, et al., AIChE Journal 22:569, 1976; Watson, J. Appl. Phys. 44:4209, 1973; Gerber, IEEE Trans. Magnetics 20:1159, 1984; Takayasu, et al., IEEE Trans. Magnetics 19:2112, 1983). In dimensionless terms, the capture cross section λ_{w}=y_{w}/R_{w} is evaluated from

$$ln {λ}_{w} = \frac{- B - \sqrt{{B}^{2} - 4 C}}{2} \qquad ( 19 )$$

where
B=−((d₁+d₂)(lnα_{w}−lnα_{w,o})+(e₁+e₂)) (20)
C=(d₁(lnα_{w}−lnα_{w,o})+e₂)(d₂(lnα_{w}−lnα_{w,o})+e₂)−c₂ (21)
c₂, d₁, d₂, e₁ and e₂ are constants in the correlation, and α_{w} is the demagnetization factor of the wire. For a ferromagnetic material of very large magnetic susceptibility at zero magnetic field strength, i.e., with χ_{w} approaching infinity, α_{w} can be expressed in terms of the magnetic saturation M_{w,s} of the wire according to

$${α}_{w} = min ( 1 , \frac{{M}_{w , s}}{2 {H}_{0}} ) \qquad ( 22 )$$

Clearly, the wire becomes magnetically saturated at a magnetic field strength that is only half the value of M_{w,s}; larger magnetic field strengths render α_{w} smaller than one. α_{w,o} is a function defined in the correlation and evaluated according to

$$ln {α}_{w , o} = \frac{- {B}_{o} - \sqrt{{B}_{o}^{2} - 4 {C}_{o}}}{2} \qquad ( 23 )$$

where
Bₒ=−((α₁+α₂)lnβ_{w}+(b₁+b₂)) (24)
Cₒ=(α₁lnβ_{w}+b₁)(α₂lnβ_{w}+b₂)−c₁ (25)

α₁, α₂, b₁, b₂, and c₂ are constants in the correlation, and β_{w} is given

$${β}_{w} = ( 1 - {ɛ}_{p} ) {ω}_{fm , p} \frac{{Re}_{w} {N}_{b} {α}_{fm , p}}{{S}^{2}} \qquad ( 26 )$$

where Re_{w} is the Reynolds number for the wire, N_{b} is the ratio between the magnetic energy of the applied magnetic field and the kinetic energy of the blood, and s is the ratio between the radius of the wire and the radius of the MDCP. These three dimensionless groups are defined as:

$$\begin{matrix}{Re}_{w} = \frac{2 {ρ}_{b} {u}_{b} {R}_{w}}{{η}_{b}} & ( 27 ) \\ {N}_{b} = \frac{{μ}_{o} {H}_{o}^{2}}{{ρ}_{b} {u}_{b}^{2}} & ( 28 ) \\ s = \frac{{R}_{w}}{{R}_{p}} & ( 29 )\end{matrix}$$

where ρ_{b} is the density of the blood, η_{b} is the viscosity of the blood, and μₒ is the permeability of free space. α_{fm,p} is the demagnetization factor of the ferromagnetic particles within the MDCPs, which are assumed to be spherical. Similar to the cylindrical wire, if the magnetic susceptibility of these spherical ferromagnetic particles is very large at zero magnetic field strength, i.e., with χ_{fm,p} approaching infinity, α_{fm,p} can be expressed in terms of the magnetic saturation M_{fm,p} of the spherical magnetic particles as

$${α}_{fm , p} = min ( 1 , \frac{{M}_{fm , p}}{3 {H}_{0}} ) \qquad ( 30 )$$

Because the ferromagnetic particles within the MDCP are spherical, α_{fm,p} takes on values of less than one only when the magnetic field strength Hₒ is greater than one-third the value of M_{fm,p}. ω_{fm,p} is the volume fraction occupied by the ferromagnetic particles in a MDCP, and εₚ is the porosity of a cluster of MDCPs if magnetic agglomeration takes place between them. The weight fraction x_{fm,p} of ferromagnetic material inside a MDCP is related to its volume fraction through

$${ω}_{fm , p} = {ρ}_{p} \frac{{x}_{fm , p}}{{ρ}_{fm , p}} \qquad ( 31 )$$

where ρ_{fm,p} is the density of the ferromagnetic material inside a MDCP and ρₚ is the average density of a MDCP. If ρ_{pol,p} represents the density of both the polymer and the drug in a MDCP, ρₚ is given by

$${ρ}_{p} = \frac{1}{\frac{{x}_{fm , p}}{{ρ}_{fm , p}} + \frac{1 - {x}_{fm , p}}{{ρ}_{pol , p}}} \qquad ( 32 )$$

The capture cross-section of the wire is evaluated from the single wire HGMS correlation (Ebner and Ritter, AIChE Journal 47:303, 2001) for the transversal configuration using Eqs. 19 to 32, the correlation constants listed in Table 2, and the physical properties and parameters given in Tables 3 and 4 for a wide range of physically realistic conditions. The resulting capture cross-sections are discussed in light of the effects of the individual elements constituting the MDT system, namely, the intensity of the magnetic field, the properties of the MDCPs, and the properties of the MIS wire. In all cases, the (dimensionless λ_{w} and/or dimensional y_{w}) capture cross section is plotted against either the magnetic field strength μₒHₒ or the blood velocity u_{b}, with the range of blood velocities being typical of that found in arteries during a systolic/diastolic heartbeat cycle (Popel, Network models of peripheral circulation, in: C. Skalak and S. Chien (Eds.), Handbook of Bioengineering, McGraw-Hill, New York, 1987, Ch 20; Berger, et al., (Eds.), Introduction to Bioengineering, Oxford University Press, New York, NY, 1996; Goldsmith and Turitto, Thrombosis and Haemistasis 55:415, 1986).

The strength of the magnetic field μₒHₒ and the velocity of the blood u_{b} are two key parameters of the MDT system that exploits the HGMS principal. μₒHₒ can be controlled to some extent, but u_{b} cannot be controlled and varies widely depending on the size and type of the blood vessel, its location in the body, and the time in the heartbeat cycle (Berger, et al., (Eds.), Introduction to Bioengineering, Oxford University Press, New York, 1996). FIG. 16 shows the effect of the blood velocity u_{b} on both the dimensionless λ_{w} and dimensional y_{w} capture cross-sections of the wire for different values of the external magnetic field strength μₒHₒ. In this case, the MIS wire has a radius of 62.5 μm (R_{w}), the MDCP has a radius of 1.0 μm (Rₚ), and both are made of 100% iron. The other physical properties and system parameters are given in Tables 3 and 4.

As one skilled in the art will appreciate, the capture cross-section consistently increases with decreasing blood velocity u_{b} and increasing magnetic field strengths μₒHₒ (FIG. 16). Further, capture cross-section is a relatively weak function of the blood velocity, increasing only moderately with decreasing u_{b}. That the capture ability of the wire does not appear to be a strong function of the blood velocity is surprising. For example, despite a 45-fold increase in u_{b} from 0.02 to 0.9 m/s, λ_{w} decreases by less than a factor of four at the highest values of μₒHₒ and by less than a factor of ten at the lowest values of μₒHₒ. These results are primarily a consequence of the strong ferromagnetic nature of the wire and the MDCPs both being comprised of iron. They are also a consequence of the short ranged character of the magnetic interactions. The strong ferromagnetic character of iron produces a very strong magnetic interaction between a MDCP and the wire that competes very well against the hydrodynamic force at close distances between them. But once the velocities become appreciable, the magnetic field strength becomes low enough, or the distance between the wire and the MDCP becomes significant, the short ranged character of the magnetic force begins to reveal itself and easily becomes dominated by any hydrodynamic force, thereby causing the capture cross-section to decrease.

Also, the capture cross-section is a strong function of the magnetic field strength μₒHₒ, increasing substantially with increasing μₒHₒ but only up to 1 T (FIG. 16). In fact, y_{w} ranging from 2 to 8 times the wire radius is easily achievable at μₒHₒ no greater than 1 T. Moreover, increasing μₒHₒ from 1 to 2 T provides only a marginal increase in y_{w}, with no further increase beyond 2 T. In consequence, it appears that the MDCPs do not require magnetic field strengths larger than about 1 T to be fully utilized. The cause of this very favorable result is again due to the strong ferromagnetic character of iron, where both the wire and the MDCPs reach magnetic saturation at around 1 T. When magnetic saturation occurs in these materials (which depends on their magnetic properties), the magnetic interaction also reaches a maximum. Hence, increasing μₒHₒ beyond some characteristic value has essentially no effect on further increasing y_{w}. In some situations y_{w} may even reach a maximum and then decrease with increasing μₒHₒ, as shown later.

The effects of the properties of a MDCP on the wire performance in terms of its size, ferromagnetic content and ferromagnetic material are shown respectively in FIGS. 17, 18, and 19. FIG. 17 shows the effect of the blood velocity u_{b} on both the dimensionless λ_{w} and dimensional y_{w} capture cross-sections of the wire for different values of the MDCP radius Rₚ. In this case, the MIS wire has a radius of 62.5 μm (R_{w}), the wire is made of iron, the MDCP is also made of 100% iron (i.e., xₚ=100 wt %), and the magnetic field strength (μₒHₒ) is 2.0 T. The MDCP with Rₚ=10 μm and porosity εₚ=0.4 is assumed to be comprised of an agglomerate of MDCPs; the ones with Rₚ<10 μm are assumed to be non-porous (i.e., εₚ=0.0), single MDCPs. The remaining parameters are given in Tables 2, 3, and 4.

The capture cross-section increases substantially with decreasing blood velocities and increasing MDCP sizes (FIG. 17). For reasonable MDCP sizes (with Rₚ ranging between 1 and 3 μm) and for flow conditions even within large arteries (with u_{b} ranging between 0.1 and 1.0 m/s), capture cross-sections ranging from 4 to 20 times the wire radius were attained. These results indicate that a single wire with R_{w}=62.5 μm can operate well in capturing MDCPs of 1 μm radius in a blood vessel that is four times the diameter of the wire when the blood velocity is around 0.2 m/s. This value approximately doubles to eight times the diameter of the wire for MDCPs of 3 μm radius, and it even triples to sixteen times the diameter of the wire for agglomerated MDCPs of 10 μm radius. Although these particular MDCPs are made of pure iron, the results are quite remarkable, especially for the 10 μm radius (agglomerated) MDCP. When substantial magnetic agglomeration of the MDCPs occurs, the application of a single wire for both collecting them at a site and directing them to a site can find many useful applications, as suggested recently (see Ritter, et al., J. Magn. Magn. Mater. 280:184-201, 2004). For the particular case studied here, it is clear that an agglomerated MDCP can be easily captured by a single wire that is operating in a very large artery of 1 mm diameter or greater and that may be experiencing blood velocities even as high as 1.0 m/s.

While not wishing to be bound by theory, it is believed that the HGMS effect also occurs between the individual MDCPs. Since the MDCPs are ferromagnetic and become polarized by an external magnetic field, they create their own magnetic field in coordination with the external one. The force generated from this localized magnetic field is sufficiently long ranged to allow attraction and retention of the MDCPs to each other. However, the factors that affect agglomeration are currently a topic of intense research (Chin, et al., Colloids and Surfaces A: Physicochemical and Engineering Aspects 204:63, 2002; Socoliuc, et al., J. Colloid Inter. Sci. 264:141, 2003; Satoh, et al., J. Colloid Inter. Sci. 209:44, 1999). A magnetically agglomerated MDCP can break up into single ones when the externally applied magnetic field is removed or its influence is out of reach. This breakup phenomenon can obviate the issue regarding agglomerated MDCPs potentially clogging capillaries located downstream due to embolization (Driscoll, et al., Microvascular Research, 27:353, 1984; Driscoll, et al., Microvascular Research, 27:353, 1984; Hafeli, Int. J. Pharm. 277:19-24, 2004).

FIG. 18 shows the effect of the blood velocity u_{b} on both the dimensionless λ_{w} and dimensional y_{w} capture cross-sections of the wire for different contents (xₚ) of ferromagnetic material in the MDCP. In this case, the MIS wire has a radius of 62.5 μm (R_{w}), the wire is made of iron, the ferromagnetic material in the MDCP is also made of iron, the MDCP has a radius of 1 μm (Rₚ), and the magnetic field strength (μₒHₒ) is 2.0 T. The remaining parameters are given in Tables 2, 3, and 4.

The capture cross-section again increases substantially with decreasing blood velocity and increasing iron content in the MDCP, with values of λ_{w} spanning from 1 to 7 at the lowest u_{b} investigated of 0.02 m/s (FIG. 18). The greater the iron content in the MDCP, the greater the magnetic force imparted on it and the greater the capture cross-section. Furthermore, even for blood velocities larger than 0.2 m/s, the ability of this wire to capture the MDCPs containing 60 wt % iron is diminished by only 60% compared to that for MDCPs containing 100 wt % iron. When considering that the iron in the 60 wt % MDCP takes up only 15% of its volume, this is surprising result because it shows that there is plenty of room in a MDCP for inclusion of the drug and polymer matrix.

Changing the ferromagnetic material in the MDCP from iron to magnetite renders similar positive results, as shown in FIG. 19. FIG. 19(a) displays the effect of the blood velocity u_{b} and FIG. 19(b) shows the effect of the magnetic field strength μₒHₒ on the dimensionless capture cross-section λ_{w} of the wire for MDCPs containing different amounts of either iron or magnetite (xₚ=40 and 100 wt %). In these cases, the MIS wire has a radius of 62.5 μm (R_{w}), the wire is made of iron, the MDCP has a radius of 1 μm (Rₚ), the magnetic field strength (μₒHₒ) is 2.0 T for the results in FIG. 19(a), and the blood velocity u_{b} is 0.3 m/s for the results in FIG. 19(b). The remaining parameters are given in Tables 2, 3, and 4.

As the blood velocity decreases and as the ferromagnetic content in the MDCP increases or became more magnetic (iron>magnetite), the capture cross-section increases substantially (FIG. 19(a)), with similar ranges and trends as just reported from the results shown in FIG. 18. However, although magnetite has a volumetric magnetic saturation of about 3.8 times smaller than iron (Table 4), the capture ability of the wire does not decrease by a factor of 3.8. At blood velocities larger than 0.2 m/s, the results in FIG. 19(a) show that the capture cross-section of the wire is reduced by only 25 to 45% when changing from magnetite to iron. This result is interesting because magnetite seems to be the ferromagnetic material of choice in the production of most MDCPs (Viroonchatapan, et al., Life Sci. 58(24):2251-2261, 1996). Magnetite is also much cheaper and more easily available than iron.

The results in FIG. 19(b) show that as the ferromagnetic material in the MDCP becomes more magnetic, the capture cross-section increases; however, as the magnetic field strength increases beyond about 1 T, the capture cross-section goes through a maximum, with values of λ_{w} in this case never exceeding 3.5. The results in FIG. 19(b) also show that at magnetic field strengths smaller than about 0.2 T, there is essentially no difference in the nature of the ferromagnetic material. Under these conditions, both of these ferromagnetic materials, which are assumed to have identical zero field magnetic susceptibilities, are not magnetically saturated. Thus, their behavior is expected to be identical when xₚ=100 wt % and only subtly different when xₚ=40 wt %, with this latter difference being due to the difference in their density, which manifests as a slight difference in the volume fraction occupied in the MDCP through Eq. 31. The consequence of the ferromagnetic material inside the MDCP becoming magnetically saturation can be very important to the design of a MDT system; hence, this topic is addressed in more detail below.

At magnetic field strengths μₒHₒ larger than one-third the value of the saturation magnetization of magnetite (i.e., at approximately 0.15 T), the spherical magnetite particles within the MDCPs become magnetically saturated (see Eq. 30). In contrast, magnetic saturation does not occur with MDCPs that contain iron until reaching a magnetic field strength μₒHₒ of about 0.58 T. This subtle difference in the magnetic saturation properties of magnetite and iron causes the slight separation in the two curves shown in FIG. 19(b) at a μₒHₒ of around 0.2 T with xₚ=0.4. Moreover, the occurrence of the maximum in the capture cross-section is a direct consequence of magnetic saturation.

At magnetic field strengths larger than about 0.87 T, in addition to the MDCPs already being magnetically saturated, the iron in the wire also becomes magnetically saturated (see Eq. 22). Under this condition, the magnetic interaction between the MDCP and the wire not only ceases to increase, but it also decreases with increasing magnetic field strengths. This phenomenon is caused by the magnetic field gradients becoming diminished (i.e., the magnetic field lines becoming straighter). This increasing (uniform) magnetic field strength μₒHₒ overwhelms the local magnetic field created by the magnetically saturated wire, which is necessarily at its maximum magnetic field strength. This overlapping of the magnetic field lines and subsequent weakening of the magnetic field gradients negatively affects the capture ability of the wire, as shown in FIG. 19(b).

The effects of the properties of the wire on its performance in terms of its size and ferromagnetic material are shown respectively in FIGS. 20 and 21. FIG. 20 shows the effect of the blood velocity u_{b} on both the dimensionless λ_{w} and dimensional y_{w} capture cross-sections of the wire for different values of the wire radius R_{w}. In this case, the wire is made of iron, the MDCP is also made of 100% iron (i.e., xₚ=100 wt %), the MDCP has a radius of 1.0 μm (Rₚ), and the magnetic field strength (μₒHₒ) is 2.0 T. The remaining parameters are given in Tables 2, 3, and 4.

The dimensionless capture cross-section λ_{w} increases with decreases in both the blood velocity u_{b} and the size of the wire R_{w}, with λ_{w} reaching as high as 10 under the most favorable conditions (i.e., with small u_{b} and small R_{w}) (FIG. 20(a)). In fact, the HGMS effect is clearly indicated from the results in FIG. 20(a), i.e., the ability of the wire to capture small MDCPs improves with smaller wires, at least when the capture cross-section is normalized to the wire radius (see below). The role of the wire size is not significant, however. Although the radius of the wire decreases by a factor of 40, at blood velocities of around 0.2 m/s, the capture ability (λ_{w}) of the wire with a radius of 25 μm is only about 7 times greater than that of the wire with a radius of 1 mm. The direct consequence of this result is that the magnetic interactions exerted by a larger wire, although weaker, are longer ranged, i.e., the MDCPs can feel the magnetic effect of the wire at farther distances away from it. This is unmistakably shown in the dimensional plot of the capture cross section shown in FIG. 20(b), where in contrast to FIG. 20(a), the role of the size of the wire is reversed.

The results in FIG. 20(b) show that as the blood velocity decreases and the size of the wire increases, the dimensional capture cross-section increases. Although this reversed role of the size of the wire seems to contradict the results in FIG. 20(b) and the corresponding HGMS effect associated with smaller wires imparting larger forces, on the contrary, it simply shows that a larger wire physically has a larger capture cross-section. But, this capture cross-section is very small relative to its size, which is the result depict in FIG. 20(a).

To further illustrate the HGMS effect in MDT, FIG. 20(b) shows that even better results can be obtained with a hypothetical wire that has a radius of “0.5 m.” A wire of this size could not be placed in an artery, but it could be placed outside the body close to the magnet and the site. This scenario makes this situation analogous to carrying out the simulation with a very large permanent magnet of high magnetic field strength but with limited magnetic field gradients and with no wire present. This situation was discussed earlier in reference to the traditional MDT approach. The correlation used in this study can simulate such a situation only by using a very large wire placed in a uniform magnetic field. This result unambiguously shows the contrast between traditional MDT, which is based on the use of an external magnet alone, and HGMS-assisted MDT, which utilizes the same magnet in cooperation with some kind of ferromagnetic article in the body like a MIS. In dimensionless terms, this large wire does not have any appreciable capture-cross section relative to its size (FIG. 20(a)); in dimensional terms, although the capture cross-section appears to be quite large, for blood velocities larger than 0.2 m/s, the capture cross section of this wire is less than 1% of its radius. This result verifies some of the claims in the literature about the limitations of traditional MDT, i.e., this large wire (or equivalently a large external magnet) is useless in targeting sites that are more than a few millimeters deep in the body (Ramchand, et al., Indian J. Pure App. Phy. 39(10):683-686, 2001; Senyei, et al., J. Appl. Phys. 49:3578, 1978; Torchilin, Eur. J. Pharm. Sci. 11, Suppl.2:S81-S91, 2000; Babincova, et al., Z. Naturforsch. C. 55(3-4):278-281, 2000; Alexiou, et al., Cancer Res. 60:6641-6648, 2000; Goodwin, et al., J. Magn. Magn. Mater. 194:132-139, 1999; Rudge, et al., J. Control. Release 74:335-340, 2001; Viroonchatapan, et al., Life Sci. 58(24):2251-2261, 1996; Gould, Materials Today 7:36-43, 2004; Lübbe, et al., J. Surgical Research 95, 200, 2001), unless the velocity is very low (<0.5 mm/s) like in arterioles and capillaries where sites as deep as 15 cm may be targeted (Hafeli, Int. J. Pharmaceutics 277:19-24, 2004.)

FIG. 21(a) shows the effect of the blood velocity u_{b} and FIG. 21(b) shows the effect of the magnetic field strength μₒHₒ on the dimensionless capture cross-section λ_{w} of the wire for wires made of different ferromagnetic materials, including Fe, 430 SS, Ni and 302 SS. In these cases, the MIS wire has a radius of 62.5 μm (R_{w}), the MDCP has a radius of 1 μm (Rₚ), the MDCP is made of 100% magnetite (xₚ=100 wt %), the magnetic field strength (μₒHₒ) is 2.0 T for the results in FIG. 21(a), and the blood velocity u_{b} is 0.3 m/s for the results in FIG. 21(b). The remaining parameters are given in Tables 2, 3, and 4.

The results in FIG. 21(a) show that the capture cross-section increases with decreasing blood velocity and increasing magnetic saturation of the wire material, which increases in the following order: 304 SS<Ni<430 SS<Fe. In contrast to the relatively moderate effect of the ferromagnetic material in the MDCPs, the capture ability of the wire depends significantly on its saturation magnetization, with Fe realizing values of λ_{w} between 1 and 5 at one extreme and 304 SS realizing values of λ_{w} only between 0 and 2 at the other extreme (both for decreasing u_{b}). In this case, the use of 304 SS, which is highly resistant to corrosion, offers little practical use for HGMS-assisted MDT. Its low magnetic saturation severely restricts the ability of the wire to capture the MDCPs. Ni is not much better. However, the use of 430 SS, which is still sufficiently corrosion resistant, at least compared to Fe, provides capture cross-sections that are only slightly less than those obtained with Fe, ranging between 1 and 4.5. This is a very promising result for HGMS-assisted MDT, because the MIS should also be corrosion resistant.

The results in FIG. 21(b) further corroborate the results discussed earlier for the MDCPs, i.e., the fact that λ_{w} exhibits a maximum with increasing μₒHₒ; but the results for the wire show more pronounced effects. For low magnetic field strengths, the capture ability of the wire is initially independent of its ferromagnetic character and increases with increasing magnetic field strength. However, as the magnetic field strength increases further, the material having the smaller saturation magnetization saturates first and so on, as explained above. Then, as μₒHₒ is increased even further, the capture ability of the wire exhibits a maximum at some magnetic field strength that depends on the magnetic saturation properties of both the wire and the MDCPs, with values of λ_{w} never exceeding 2 in the best case scenario for these particular conditions. In fact, this optimum behavior appears to be a general result for all ferromagnetic materials. With reference to the system studied here, this very positive result once again suggests that magnetic field strengths no larger than about 1.0 T are required to operate a HGMS assisted MDT system. This result also means that there is a compromise between the type of ferromagnetic material used to make the wire, the type of ferromagnetic material used to make the MDCPs, and the source of the external magnetic field. Clearly, the external magnetic field source can be chosen such that its intensity maximizes the capture ability of the MIS, which in turn depends on the ferromagnetic character of both the MDCPs and the MIS.

The use of a biocompatible article comprising a magnetizable intravascular stent (MIS) as part of a magnetic drug targeting (MDT) system is disclosed herein. This MDT system comprises magnetic drug carrier particles (MDCPs), an external magnetic field source, and the MIS of ferromagnetic nature that has been implanted in a blood vessel adjacent to the target site. The MDT approach disclosed herein exploits the use of high gradient magnetic separations (HGMS) principles through the MIS to vastly improve the retention of the MDCPs at the target site.

The performance of the exemplified MDT system was examined in terms of the ability of one of the wires in the MIS to capture the MDCPs, with the capture cross-section evaluated from a single wire HGMS correlation in the literature that assumes the wire to be perpendicular to both the flow and the external magnetic field in a transversal configuration, the blood and MDCPs to be free from wall effects, and the blood to be under potential flow. A parametric study showed that the dimensionless capture cross section (with respect to the wire radius) increases with lower blood velocities (0.02 to 0.9 m/s), higher applied magnetic field strengths (0.2 to 2.0 T), larger MDCPs (0.2 to 10 μm radius) containing more (10 to 100%) and stronger (iron or magnetite) ferromagnetic material, and smaller wires (20 to 150 μm in radius) comprised of stronger ferromagnetic material (iron>430 SS>nickel>304 SS).

Capture cross-sections between 2 and 3, but as high as 12, times the radius of the wire were easily attained for just a single wire and under the extreme flow conditions of 0.9 m/s that are typical of large arteries in the circulatory system. These results are even more encouraging when considering that an actual MIS has multiple wires, the recirculation period of the circulatory system is quite short, and wires of almost any size comparable to that of the blood vessels can be used.

The results from this correlation also provided considerable insight to the proper design of a MDT system. For example, the results verified that target sites more than a few centimeters deep in the body cannot be reached with the traditional MDT approach, which utilizes only an external magnetic field to effect capture of the MDCPs at the site. The results also indicated that magnetic field strengths of around 1 T should suffice for any HGMS-based MDT approach.

TABLE 2
Parameters for the single wire HGMS capture cross-section correlation
under the transversal configuration
(Ebner and Ritter, AIChE Journal 47: 303, 2001).

| Parameter | Value |
|---|---|
| a₁ | −0.48990 |
| a₂ | −1.02248 |
| b₁ | 0.52197 |
| b₂ | 1.50099 |
| c₁ | 0.36778 |
| c₂ | 1.66374 |
| d₁ | 0.34487 |
| d₂ | 2.07542 |
| e₁ | 0.77117 |
| e₂ | 2.07217 |

TABLE 3
Values and ranges of the physical parameters used in the single wire
HGMS capture cross-section correlation for the parametric study.

| | | |
|---|---|---|
| Properties | Units | Value(s) |

Blood

| | | |
|---|---|---|
| ρ_{b} | kg/m³ | 1040 |
| η_{b} | kg/(m s) | 3.0 × 10⁻³ |
| χ_{b} | SI | 0 |
| U_{b} | m s⁻¹ | 0.02-0.90 |

Drug Carrier

| | | |
|---|---|---|
| ^{a}material | — | Fe, Fe₃O₄ |
| ρ_{pol,p} | kg m⁻³ | 950 |
| χ_{,pol,p} | SI | 0 |
| X_{fm,p} | % | 10, 40, 60, 100 |
| ^{b}Rₚ | μm | 0.2, 0.5, 1.0, 3.0, 10.0 |
| ^{b}εₚ | — | 0.0, 0.4 |
| Wire | | |
| ^{a}material | — | Fe, 430, Ni, 304 |
| R_{w} | μm | 25, 62.5, 150 |

Magnetic Field

| | | |
|---|---|---|
| β | — | π/2, 0 |
| μₒHₒ | T | 0.05-1.0 |

^{a}magnetic material with properties provided in Table 3
^{b}εₚ = 0 for Rₚ ≦ 3.0 μm and εₚ = 0.4 for Rₚ > 3.0 μm

TABLE 4
Physical properties of various types of ferromagnetic materials
used in the single wire HGMS capture cross-section correlation
for the parametric study.

| | Density | Mₛₐₜ | Mₛₐₜ |
|---|---|---|---|
| Material^{a} | (g/cc) | (emu/g) | (kA/m) |
| Iron | 7.85 | 221 | 1735 |
| 304 SS | 8.00 | 20 | 160 |
| 430 SS | 7.66 | 165 | 1264 |
| Nickel | 8.91 | 55 | 490 |
| Fe₃O₄ | 5.05 | 90 | 455 |

^{a}All materials are assumed to have a zero magnetic field susceptibility (χ_{fm,p}) of 100 (SI).

### Specific Embodiments

Disclosed herein, in one aspect, is an article that is reactive to an external magnetic field comprising a magnetizable member, wherein the magnetizable member produces a magnetic force density of from about 1×10⁴ to about 1×10¹⁴ N/m³ when placed under the influence of an external magnetic field with a strength of from about 1 to about 8000 kA/m. Also disclosed, in another aspect, is an article that is reactive to an external magnetic field comprising a magnetizable member, wherein the magnetizable member comprises from about 50 to about 100% by weight of the article of a magentizable material, and wherein the magnetizable member produces a magnetic force density of from about 1×10⁴ to about 1×10¹⁴ N/m³ when placed under the influence of an external magnetic field with a strength of from about 1 to about 8000 kA/m.

In another aspect, disclosed herein is a therapeutic treatment system comprising a magnetic field generator and an article, wherein the article comprises a magnetizable member and wherein the magnetizable member becomes magnetic when placed within a field generated by the magnetic field generator. The system can further comprise a magnetic drug carrier particle.

In further aspects, disclosed herein is a method of treating a disease or disorder in a subject by placing an article within the body of the subject, wherein the article comprises a magnetizable member, inserting a magnetic drug carrier particle comprising a drug into the body of the subject, and applying a magnetic field to the article, thereby causing the magnetic drug carrier particle to be attracted to a zone near the article where the activity of the drug is expressed. Also disclosed is a method of treating a disease or disorder in a subject by placing an article adjacent to the skin of the subject near a diseased site, wherein the article comprises a magnetizable member, inserting a magnetic drug carrier particle comprising a drug into the body of the subject, and applying a magnetic field to the article, thereby causing the magnetic drug carrier particle to be attracted to a zone near the article where the activity of the drug is expressed. Still further, disclosed is a method of treating restenosis in a subject by placing a magnetizable wire next to a part of an artery of the subject that is to be treated for restenosis, inserting a magnetic drug carrier particle comprising a drug having activity against restenosis in the artery, and applying a magnetic field to the wire, thereby causing the magnetic drug particle to be attracted to a zone within the artery and adjacent the wire where the activity of the drug is expressed. Also disclosed is a method of positioning a magnetic drug carrier particle within the body of a subject, the method comprising placing an article within the body of the subject or external to the body of a subject, wherein the article comprises a magnetizable member, inserting a magnetic drug carrier particle into the body of the subject, and applying an external magnetic field to the article, thereby causing the magnetic drug carrier particle to be attracted to the article.

In yet another aspect, disclosed herein is a kit for positioning a magnetic drug carrier particle within the body of a subject, the kit comprising: a magnetizable member; and a magnetic drug carrier particle.

As illustrated in the following examples, the magnetizable member can produce a magnetic force density of from about 1×10⁴ to about 1×10¹⁴ N/m³ when placed under the influence of an external magnetic field with a strength of from about 1 to about 8000 kA/m. The magentizable member can become heated when placed within an alternating field generated by the magnetic field generator. The magnetizable member can produce substantially zero field in the absence of the external magnetic field, e.g., can be substantially non-magnetic when not under the external magnetic field. The magnetizable member can be paramagnetic. The magnetizable member can be ferromagnetic. The magnetizable member can be anti-ferromagnetic. The magnetizable member can be ferrimagnetic. The magnetizable member can be superparamagnetic. The magnetizable member can comprise magnetic stainless steel. The magnetizable member can comprise a composite material. The magnetizable member can comprise a magnetizable material. The magnetizable material can be present in an amount of from about 50 to about 100% by weight of the article.

In further examples, the article can comprise a seed. The seed can have diameter of from 1 to about 2000 nanometers. The seed can have a diameter of about 10 to about 2000 nanometers. The seed can have diameter of from 1 to about 1000 nanometers. The seed can have a diameter of from 2 to about 500 nanometers. The seed can have a diameter of from 50 to about 200 nanometers. The seed can have a diameter of less than about 1000 nanometers, or less than about 100 nanometers. The seed can be sufficiently small as to pass through human capillaries without clogging them. The seed can be round, oblong, square, rectangular, irregular, cylindrical, spiral, toroidal, ring, spherical, or plate-like in shape. The article can also comprise a plurality of seeds, wherein the plurality of seeds comprises an agglomeration. The article can comprise one or more wires. The article can comprise one or more stents. The article can comprise one or more needles. The article can comprise one or more catheters or one or more catheter tips. The article can comprise one or more coils, meshes, or beads. The article can be adapted to be positioned within a subject. The article can be adapted to be positioned near a subject. The article can be adapted to be removed from a subject.

In still other examples, the magnetic field generator can comprise a permanent magnet. The magnetic field generator can comprise an electromagnet. The magnetic field generator can comprise a superconducting magnet. The magnetic field generator can be a magnet that is located external to the body of the subject. The magnetic field generator can have a field strength sufficient to position the magnetic drug carrier particle.

In yet other examples, the magnetic drug carrier particle can comprise a pharmaceutical composition. The magnetic drug carrier particle can comprise a radioactive composition. The magnetic drug carrier particle can comprise a vesicle, polymer, metal, mineral, protein, lipid, carbohydrate, or mixture thereof. The magnetic drug carrier particle can comprise a plurality of particles having an average diameter of from about 10 to about 2000 nanometers. The magnetic drug carrier particle can have diameter of from 1 to about 1000 nanometers. The magnetic drug carrier particle can have a diameter of from 2 to about 500 nanometers. The magnetic drug carrier particle can have a diameter of from 50 to about 200 nanometers. The magnetic drug carrier particle can have a diameter of less than about 1000 nanometers, or less than about 100 nanometers. The magnetic drug carrier particle can comprise a paramagnetic, ferromagnetic, anti-ferromagnetic, ferrimagnetic, or superparamagnetic material. The magnetic drug carrier particle can comprise magnetite. The magnetic drug carrier particle can comprise magnetite in an amount from about 1 to about 98% by weight of the particle. The magnetic drug carrier particle can comprise magnetite in an amount from about 5 to about 95% by weight of the particle. The magnetic drug carrier particle can comprise magnetite in an amount from about 10 to about 90% by weight of the particle. The magnetic drug carrier particle can comprise magnetite in an amount from about 30 to about 80% by weight of the particle.

In the disclosed methods, placing can comprise placing the article adjacent to the skin of the subject. The skin can be near a diseased site. Placing can comprise implanting the article transdermally within the body of the subject. Placing can comprise placing the article at a location within the body of the subject that is adjacent to a diseased site. Placing can comprise placing the article at a location within the body of the subject that is adjacent to a blood vessel. Placing can comprise placing the article at a location within the body of the subject that is adjacent to a carotid bifurcation. Placing can comprise injecting the article into the body of the subject and positioning the article at a target site. The article can be injected into the blood circulation system of the subject. The article can be positioned at the targeted site by applying a magnetic field to the body of the subject at a location that causes the article to move to the targeted site. The targeted site can be sufficiently deep under the skin of the subject that an external magnetic field alone cannot provide sufficient power to retain particles at the targeted site. Inserting the magnetic drug carrier particle can comprise injecting the magnetic drug carrier particle into the body of the subject. The magnetic drug carrier particle can be injected into the blood circulation system of the subject. The magnetic drug carrier particle can be injected into the body of the subject at the same time as the article. Applying an external magnetic field can comprise positioning a permanent magnet so that the article is within its magnetic field. Applying an external magnetic field can comprise positioning an electromagnet so that the article is within its magnetic field. Applying an external magnetic field can comprise positioning a superconducting magnet so that the article is within its magnetic field. Applying an external magnetic field can comprise providing a magnetic field at a location that includes the article and having a field strength sufficient to position the magnetic drug carrier particle. The magnetic field can have a strength of from about 1 to about 8000 kA/m. The magnetic field can have a strength of from about 1 to about 800 kA/m. The magnetic field can have a strength of from about 1 to about 80 kA/m.

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

What is claimed is:

1. An article that is reactive to an external magnetic field, comprising: a magnetizable member, wherein the magnetizable member produces a magnetic force density of from about 1×10⁴ to about 1×10¹⁴ N/m³ when placed under the influence of an external magnetic field with a strength of from about 1 to about 8000 kA/m.

2. The article of claim 1, wherein the magnetizable member produces substantially zero field in the absence of the external magnetic field.

3. The article of claim 1, wherein the magnetizable member is paramagnetic.

4. The article of claim 1, wherein the magnetizable member is ferromagnetic.

5. The article of claim 1, wherein the magnetizable member is anti-ferromagnetic.

6. The article of claim 1, wherein the magnetizable member is ferrimagnetic.

7. The article of claim 1, wherein the magnetizable member is superparamagnetic.

8. The article of claim 1, wherein the magnetizable member comprises magnetic stainless steel.

9. The article of claim 1, wherein the magnetizable member comprises a composite material.

10. The article of claim 1, wherein the article comprises a seed.

11. The article of claim 10, wherein the seed has a diameter of from about 1 to about 2000 nanometers.

12. The article of claim 10, wherein the seed is sufficiently small as to pass through human capillaries without clogging them.

13. The article of claim 10, wherein the seed is round, oblong, square, rectangular, irregular, cylindrical, spiral, toroidal, ring, spherical, or plate-like in shape.

14. The article of claim 10, wherein the article comprises a plurality of seeds and wherein the plurality of seed comprises an agglomeration.

15. The article of claim 1, wherein the article comprises one or more wires.

16. The article of claim 1, wherein the article comprises one or more stents.

17. The article of claim 1, wherein the article comprises one or more needles.

18. The article of claim 1, wherein the article comprises one or more catheters or one or more catheter tips.

19. The article of claim 1, wherein the article comprises one or more coils, meshes, or beads.

20. The article of claim 1, wherein the magnetizable member comprises a magnetizable material.

21. The article of claim 20, wherein magnetizable material is present in an amount of from about 50 to about 100% by weight of the article.

22. An article that is reactive to an external magnetic field, comprising: a magnetizable member, wherein the magnetizable member comprises from about 50 to about 100% by weight of the article of a magentizable material, and wherein the magnetizable member produces a magnetic force density of from about 1×10⁴ to about 1×10¹⁴ N/m³ when placed under the influence of an external magnetic field with a strength of from about 1 to about 8000 kA/m.

23. The article of claim 22, wherein the magnetizable member is substantially non-magnetic when not under the external magnetic field.

24. A therapeutic treatment system, comprising:
a. a magnetic field generator; and
b. an article, wherein the article comprises a magnetizable member and wherein the magnetizable member becomes magnetic when placed within a field generated by the magnetic field generator.

25. The system of claim 24, wherein the magnetizable member produces a magnetic force density of from about 1×10⁴ to about 1×10¹⁴ N/m³ when placed under the influence of an external magnetic field with a strength of from about 1 to about 8000 kA/m.

26. The system of claim 24, wherein the magentizable member becomes heated when placed within an alternating field generated by the magnetic field generator.

27. The system of claim 24, wherein the magnetic field generator comprises a permanent magnet.

28. The system of claim 24, wherein the magnetic field generator comprises an electromagnet.

29. The system of claim 24, wherein the magnetic field generator comprises a superconducting magnet.

30. The system of claim 24, wherein the magnetizable member is paramagnetic.

31. The system of claim 24, wherein the magnetizable member is ferromagnetic.

32. The system of claim 24, wherein the magnetizable member is anti-ferromagnetic.

33. The system of claim 24, wherein the magnetizable member is ferrimagnetic.

34. The system of claim 24, wherein the magnetizable member is superparamagnetic.

35. The system of claim 24, wherein the magnetizable member comprises magnetic stainless steel.

36. The system of claim 24, wherein the magnetizable member comprises a composite material.

37. The system of claim 24, wherein the article comprises a seed.

38. The system of claim 37, wherein the seed has a diameter of from about 1 to about 2000 nanometers.

39. The system of claim 37, wherein the seed is sufficiently small as to pass through human capillaries without clogging them.

40. The system of claim 37, wherein the seed is round, oblong, square, rectangular, irregular, cylindrical, spiral, toroidal, ring, spherical, or plate-like in shape.

41. The system of claim 24, wherein the article comprises a plurality of seeds and wherein the plurality of seed comprises an agglomeration.

42. The system of claim 24, wherein the article comprises one or more wires.

43. The system of claim 24, wherein the article comprises one or more stents.

44. The system of claim 24, wherein the article comprises one or more needles.

45. The system of claim 24, wherein the article comprises one or more catheters or article comprises one or more catheter tips.

46. The system of claim 24, wherein the article comprises one or more coils, meshes, or beads.

47. The system of claim 24, wherein the magnetizable member comprises a magnetizable material.

48. The system of claim 47, wherein magnetizable material is present in an amount of from about 50 to about 100% by weight of the article.

49. The system of claim 24, wherein the article is adapted to be positioned within a subject.

50. The system of claim 24, wherein the article is adapted to be positioned near a subject.

51. The system of claim 24, wherein the article is adapted to be removed from a subject.

52. The system of claim 24, further comprising a magnetic drug carrier particle.

53. The system of claim 52, wherein the magnetic drug carrier particle comprises a pharmaceutical composition.

54. The system of claim 52, wherein the magnetic drug carrier particle comprises a radioactive composition.

55. The system of claim 52, wherein the magnetic drug carrier particle comprises a vesicle, polymer, metal, mineral, protein, lipid, carbohydrate, or mixture thereof.

56. The system of claim 52, wherein the magnetic drug carrier particle comprises a plurality of particles having an average diameter of from about 10 to about 2000 nanometers.

57. The system of claim 52, wherein the magnetic drug carrier particle comprises a paramagnetic, ferromagnetic, anti-ferromagnetic, ferrimagnetic, or superparamagnetic material.

58. The system of claim 52, wherein the magnetic drug carrier particle comprises magnetite.

59. The system of claim 52, wherein the magnetic drug carrier particle comprises magnetite in an amount from about 1 to about 98% by weight of the particle.

60. A method of positioning a magnetic drug carrier particle within the body of a subject, the method comprising:
a. placing an article within the body of the subject or external to the body of a subject, wherein the article comprises a magnetizable member;
b. inserting a magnetic drug carrier particle into the body of the subject; and
c. applying an external magnetic field to the article, thereby causing the magnetic drug carrier particle to be attracted to the article.

61. The method of claim 60, wherein the magnetic drug carrier particle comprises pharmaceutical composition.

62. The method of claim 60, wherein the magnetic drug carrier particle comprises a radioactive composition.

63. The method of claim 60, wherein the magnetic drug carrier particle comprises a vesicle, polymer, metal, mineral, protein, lipid, carbohydrate, or mixture thereof.

64. The method of claim 60, wherein the magnetic drug carrier particle comprises a plurality of particles having an average diameter of from about 10 to about 2000 nanometers.

65. The method of claim 60, wherein the magnetic drug carrier particle comprises a paramagnetic, ferromagnetic, anti-ferromagnetic, ferrimagnetic, or superparamagnetic material.

66. The method of claim 60, wherein the magnetic drug carrier particle comprises magnetite.

67. The method of claim 66, wherein the magnetic drug carrier particle comprises magnetite in an amount from about 1 to about 98% by weight of the particle.

68. The method of claim 60, wherein the magnetizable member is paramagnetic.

69. The method of claim 60, wherein the magnetizable member is ferromagnetic.

70. The method of claim 60, wherein the magnetizable member is anti-ferromagnetic.

71. The method of claim 60, wherein the magnetizable member is ferrimagnetic.

72. The method of claim 60, wherein the magnetizable member is superparamagnetic.

73. The method of claim 60, wherein the magnetizable member comprises magnetic stainless steel.

74. The method of claim 60, wherein the magnetizable member comprises a composite material.

75. The method of claim 60, wherein the article comprises a seed.

76. The method of claim 75, wherein the seed has a diameter of from about 1 to about 2000 nanometers.

77. The method of claim 75, wherein the seed is sufficiently small as to pass through human capillaries without clogging them.

78. The method of claim 75, wherein the seed is round, oblong, square, rectangular, irregular, cylindrical, spiral, toroidal, ring, spherical, or plate-like in shape.

79. The method of claim 60, wherein the article comprises a plurality of seeds and wherein the plurality of seed comprises an agglomeration.

80. The method of claim 60, wherein the article comprises one or more wires.

81. The method of claim 60, wherein the article comprises one or more stents.

82. The method of claim 60, wherein the article comprises one or more needles.

83. The method of claim 60, wherein the article comprises one or more catheters or article comprises one or more catheter tips.

84. The method of claim 60, wherein the article comprises one or more coils, meshes, or beads.

85. The method of claim 60, wherein the magnetizable member comprises a magnetizable material.

86. The method of claim 85, wherein magnetizable material is present in an amount of from about 50 to about 100% by weight of the article.

87. The method of claim 60, wherein placing comprises placing the article adjacent to the skin of the subject.

88. The method of claim 87, wherein the skin is near a diseased site.

89. The method of claim 60, wherein placing comprises implanting the article transdermally within the body of the subject.

90. The method of claim 60, wherein placing comprises placing the article at a location within the body of the subject that is adjacent to a diseased site.

91. The method of claim 60, wherein placing comprises placing the article at a location within the body of the subject that is adjacent to a blood vessel.

92. The method of claim 60, wherein placing comprises placing the article at a location within the body of the subject that is adjacent to a carotid bifurcation.

93. The method of claim 60, wherein placing comprises injecting the article into the body of the subject and positioning the article at a target site.

94. The method of claim 93, wherein the article is injected into the blood circulation system of the subject.

95. The method of claim 93, wherein the article is positioned at the targeted site by applying a magnetic field to the body of the subject at a location that causes the article to move to the targeted site.

96. The method of claim 93, wherein the targeted site is sufficiently deep under the skin of the subject that an external magnetic field alone cannot provide sufficient power to retain particles at the targeted site.

97. The method of claim 60, wherein in inserting the magnetic drug carrier particle comprises injecting the magnetic drug carrier particle into the body of the subject.

98. The method of claim 97, wherein the magnetic drug carrier particle is injected into the blood circulation system of the subject.

99. The method of claim 97, wherein the magnetic drug carrier particle is injected into the body of the subject at the same time as the article.

100. The method of claim 60, wherein applying an external magnetic field comprises positioning a permanent magnet so that the article is within its magnetic field.

101. The method of claim 60, wherein applying an external magnetic field comprises positioning an electromagnet so that the article is within its magnetic field.

102. The method of claim 60, wherein applying an external magnetic field comprises positioning a superconducting magnet so that the article is within its magnetic field.

103. The method of claim 60, wherein applying an external magnetic field comprises providing a magnetic field at a location that includes the article and having a field strength sufficient to position the magnetic drug carrier particle.

104. The method of claim 60, wherein the magnetic field has a strength of from about 1 to about 8000 kA/m.

105. The method of claim 60, wherein the magnetic field has a strength of from about 1 to about 800 kA/m.

106. The method of claim 60, wherein the magnetic field has a strength of from about 1 to about 80 kA/m.

107. A method of treating a disease or disorder in a subject, the method comprising:
a. placing an article within the body of the subject, wherein the article comprises a magnetizable member;
b. inserting a magnetic drug carrier particle comprising a drug into the body of the subject; and
c. applying a magnetic field to the article, thereby causing the magnetic drug carrier particle to be attracted to a zone near the article where the activity of the drug is expressed.

108. A method of treating a disease or disorder in a subject, the method comprising:
a. placing an article adjacent to the skin of the subject near a diseased site, wherein the article comprises a magnetizable member;
b. inserting a magnetic drug carrier particle comprising a drug into the body of the subject; and
c. applying a magnetic field to the article, thereby causing the magnetic drug carrier particle to be attracted to a zone near the article where the activity of the drug is expressed.

109. A kit for positioning a magnetic drug carrier particle within the body of a subject, the kit comprising: a magnetizable member; and a magnetic drug carrier particle.

110. The kit of claim 109, wherein the magnetic drug carrier particle comprises pharmaceutical composition.

111. The kit of claim 109, wherein the magnetic drug carrier particle comprises radioactive composition.

112. The kit of claim 109, wherein the magnetic drug carrier particle comprises a vesicle, polymer, metal, mineral, protein, lipid, carbohydrate, or mixture thereof.

113. The kit of claim 109, wherein the magnetic drug carrier particle comprises a plurality of particles having an average diameter of from about 10 to about 2000 nanometers.

114. The kit of claim 109, wherein the magnetic drug carrier particle comprises a paramagnetic, ferromagnetic, anti-ferromagnetic, ferrimagnetic, or superparamagnetic material.

115. The kit of claim 109, wherein the magnetic drug carrier particle comprises magnetite.

116. The kit of claim 114, wherein the magnetic drug carrier particle comprises magnetite in an amount from about 1 to about 98% by weight of the particle.

117. The kit of claim 114, wherein the magnetizable member is paramagnetic.

118. The kit of claim 114, wherein the magnetizable member is ferromagnetic.

119. The kit of claim 114, wherein the magnetizable member is anti-ferromagnetic.

120. The kit of claim 114, wherein the magnetizable member is ferrimagnetic.

121. The kit of claim 114, wherein the magnetizable member is superparamagnetic.

122. The kit of claim 114, wherein the magnetizable member comprises magnetic stainless steel.

123. The kit of claim 114, wherein the magnetizable member comprises a composite material.

124. The kit of claim 114, wherein the magnetizable member comprises a seed.

125. The kit of claim 124, wherein the seed has a diameter of from about 1 to about 2000 nanometers.

126. The kit of claim 124, wherein the seed is sufficiently small as to pass through human capillaries without clogging them.

127. The kit of claim 124, wherein the seed is round, oblong, square, rectangular, irregular, cylindrical, spiral, toroidal, ring, spherical, or plate-like in shape.

128. The kit of claim 114, wherein the article comprises a plurality of seeds and wherein the plurality of seed comprises an agglomeration.

129. The kit of claim 114, wherein the magnetizable member comprises one or more wires.

130. The kit of claim 114, wherein the magnetizable member comprises one or more stents.

131. The kit of claim 114, wherein the magnetizable member comprises one or more needles.

132. The kit of claim 114, wherein the magnetizable member comprises one or more catheters or article comprises one or more catheter tips.

133. The kit of claim 114, wherein the magnetizable member comprises one or more coils, meshes, or beads.

134. The kit of claim 114, wherein the magnetizable member comprises a magnetizable material.

135. The kit of claim 134, wherein magnetizable material is present in an amount of from about 50 to about 100% by weight of the article.

136. The kit of claim 109, further comprising a magnetic field generator.

137. The kit of claim 136, wherein the magnetic field generator is a magnet that is located external to the body of the subject.

138. The kit of claim 136, wherein the magnetic field generator comprises a permanent magnet.

139. The kit of claim 136, wherein the magnetic field generator comprises an electromagnet.

140. The kit of claim 136, wherein the magnetic field generator comprises a superconducting magnet.

141. The kit of claim 136, wherein the magnetic field generator has a field strength sufficient to position the magnetic drug carrier particle.
